(19) United States
(12) Patent Application Publication
(10) Pub. No.: US 2025/0339258 A1
(43) Pub. Date: Nov. 6, 2025

(54) COMPLIANT LOBE-SHAPED IMPLANT DEVICES

(71) Applicant: EDWARDS LIFESCIENCES CORPORATION, Irvine, CA (US)

(72) Inventors: Leonardo Paim Nicolau Da Costa, Tampa, FL (US); Emil Karapetian, Huntington Beach, CA (US)

(21) Appl. No.: 19/268,406

(22) Filed: Jul. 14, 2025

Related U.S. Application Data
(63) Continuation of application No. PCT/US2024/012833, filed on Jan. 24, 2024.
(60) Provisional application No. 63/481,952, filed on Jan. 27, 2023.

(51) Int. Cl. A61F 2/07; A61F 2/24

(52) U.S. Cl. CPC A61F 2/07; A61F 2/2475; A61F 2002/072; A61F 2210/0014

(57) ABSTRACT

Devices, systems, and/or methods can treat aneurysms and/or other conditions, while providing compliance characteristics to fluid vessels. Examples of the present disclosure can include an implant device that includes a tubular structure with multiple first sections that deflect radially inward and that are separated by multiple second sections. The second sections can include lobes. The first sections can be adapted to deflect radially outward based on radial/luminal pressure within a fluid vessel to provide a change in volume for the implant device.

### RELATED APPLICATION(S)

This application is a continuation of International Patent Application No. PCT/US2024/012833, filed Jan. 24, 2024, which claims the benefit of U.S. Provisional Patent Application No. 63/481,952, filed on Jan. 27, 2023, the complete disclosures of which are hereby incorporated by reference in their entireties.

### BACKGROUND

The present disclosure generally relates to the field of medical devices and methods for vascular repair. Aneurysms are permanent dilations in blood vessel walls due to weakened or abnormal tissue. In some instances, an aneurysm can rupture, causing internal bleeding that presents a serious risk to the patient, such as death. Aneurysms can occur in various parts of the body, including the aorta, brain, and elsewhere. Further, the aorta and other blood vessels are affected by other conditions that adversely affect the function of the blood vessels.

### SUMMARY

Described herein are devices, methods, and/or systems that treat aneurysms and other conditions in blood vessels while providing compliance characteristics. For example, devices of the present disclosure can include a tubular structure with a cross-sectional shape having multiple first sections that deflect radially inward and are separated by multiple second sections. The multiple first sections can include one or more compliant characteristics. The device can also include an attachment feature coupled to or integral with the tubular structure and configured to couple to a fluid vessel. The multiple first section can be configured to expand and contract radially based on fluid pressure within the fluid vessel to provide a change in volume. The change in volume can allow the blood vessel to mimic compliance of a healthy blood vessel and/or otherwise promote blood flow during, for example, a phase of the cardiac cycle.

For purposes of summarizing the disclosure, certain aspects, advantages, and/or features are described. It is to be understood that not necessarily all such aspects/advantages may be achieved in accordance with any particular example. Thus, the disclosed examples can be carried out in a manner that achieves or optimizes one advantage or group of advantages as taught hercin without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIG. 1A illustrates an example representation of a heart and associated vasculature having various features relevant to one or more examples of the present disclosure.

FIGS. 1B-1 illustrates an example healthy aorta.

FIGS. 1B-2 illustrates an example unhealthy aorta.

FIG. 2A illustrate a side view of an example implant device/system configured to be implanted/disposed at a target site and provide compliant characteristics to a fluid vessel according to one or more examples.

FIG. 2B illustrates a first perspective view of the implant device/system of FIG. 2A.

FIG. 2C illustrates a second perspective view of the implant device/system of FIG. 2A.

FIG. 3 illustrates an example implant device with attachment segment(s) configured to expand according to one or more examples.

FIG. 4A-1 illustrates an example implant device in a relaxed/default/non-expanded/unpressurized state according to one or more examples.

FIG. 4A-2 illustrates a cross-sectional view through the implant device in the configuration of FIG. 4A-1 according to one or more examples.

FIG. 4B-1 illustrates an example implant device in an expanded/non-relaxed state according to one or more examples.

FIG. 4B-2 illustrates a cross-sectional view through the implant device in the configuration of FIG. 4B-1 according to one or more examples.

FIG. 5 illustrates an example implant device configured with different properties/characteristics for deflectable portions and lobes according to one or more examples.

FIG. 6 illustrates an example implant device with a prosthetic valve according to one or more examples.

FIG. 7A illustrates an example implant device with four deflectable portions and lobes according to one or more examples.

FIG. 7B illustrates a cross-sectional view of the implant device of FIG. 7A.

FIG. 8 illustrates an example implant device implanted at a resected portion of the aorta according to one or more examples.

FIG. 9 illustrates an example implant device implanted within an aneurysmal section of the aorta that is not resected according to one or more examples.

FIG. 10 an example implant device implanted within a section of the aorta that is free of an aneurysm according to one or more examples.

FIG. 11 illustrates a flow diagram of a process for implanting a device using a surgical procedure in accordance with one or more examples.

FIG. 12 illustrates a flow diagram of a process for implanting a device using an endovascular/minimally invasive procedure in accordance with one or more examples.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for ease of description to describe the positional relationship between element(s)/structures(s), as illustrated in the drawings. Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

### Vascular Anatomy

Certain examples are disclosed herein in the context of vascular implant devices, and in particular, compliance implant devices implanted in the aorta. However, although certain principles disclosed herein can be particularly applicable to the anatomy of the aorta, the compliance implant devices in accordance with the present disclosure can be implanted in, or configured for implantation in, any suitable or desirable blood vessels or other anatomy, such as the inferior vena cava, etc.

The anatomy of the heart and vascular system is described below to assist in the understanding of certain concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles can be prompted by signals generated by the electrical system of the heart.

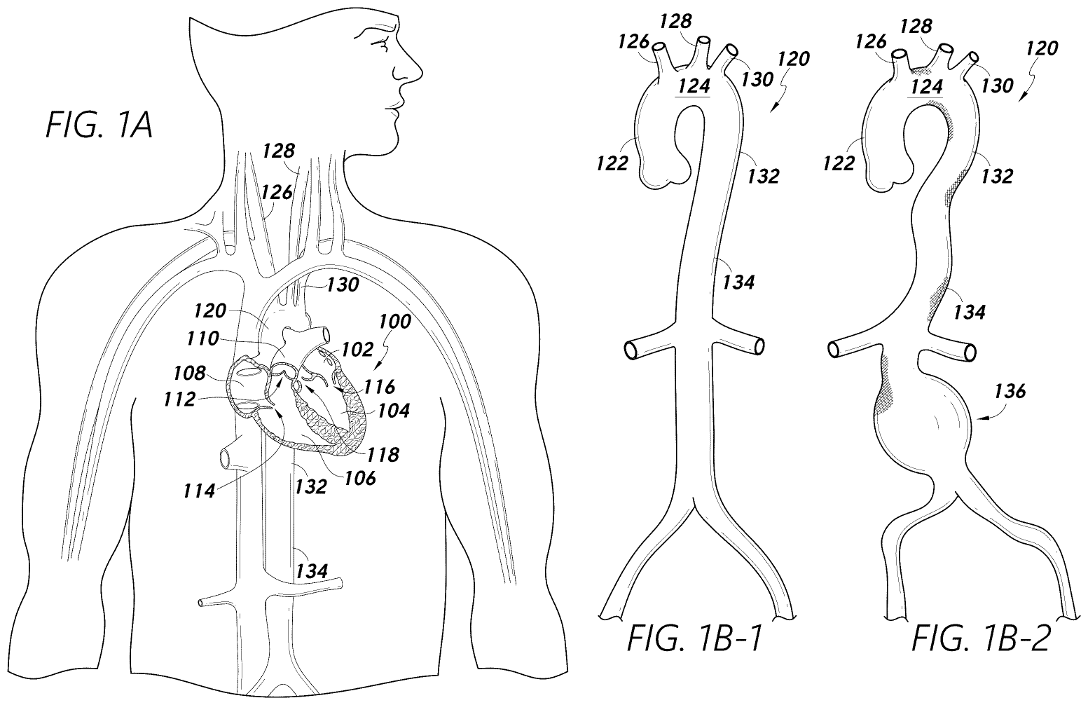

FIG. 1A illustrates an example representation of a heart 100 and associated vasculature having various features relevant to one or more examples of the present disclosure. The heart 100 includes four chambers, namely the left atrium 102, the left ventricle 104, the right ventricle 106, and the right atrium 108. In terms of blood flow, blood generally flows from the right ventricle 106 into the pulmonary artery 110 via the pulmonary valve 112, which separates the right ventricle 106 from the pulmonary artery 110 and is configured to open during systole so that blood can be pumped toward the lungs and close during diastole to prevent blood from leaking back into the heart from the pulmonary artery 110. The pulmonary artery 110 carries deoxygenated blood from the right side of the heart 100 to the lungs. The pulmonary artery 110 includes a pulmonary trunk and left and right pulmonary arteries that branch off the pulmonary trunk, as shown.

The tricuspid valve 114 separates the right atrium 108 from the right ventricle 106. The tricuspid valve 114 generally has three cusps/leaflets and can generally close during ventricular contraction (i.e., systole) and open during ventricular expansion (i.e., diastole). The mitral valve 116 generally has two cusps/leaflets and separates the left atrium 102 from the left ventricle 104. The mitral valve 116 is configured to open during diastole so that blood in the left atrium 102 can flow into the left ventricle 104, and, when functioning properly, closes during systole to prevent blood from leaking back into the left atrium 102. The aortic valve 118 separates the left ventricle 104 from the aorta 120. The aortic valve 118 is configured to open during systole to allow blood leaving the left ventricle 104 to enter the aorta 120, and close during diastole to prevent blood from leaking back into the left ventricle 104.

The heart valves can generally comprise a relatively dense fibrous ring, referred to herein as the annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, can generally comprise finger-like projections from the ventricle wall. The valve leaflets are connected to the papillary muscles by the chordae tendineae. A wall of muscle, referred to as the septum, separates the left 102 and right 108 atria and the left 104 and right 106 ventricles.

The vasculature of the human body, which can be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior venae cavae, carry blood back to the heart.

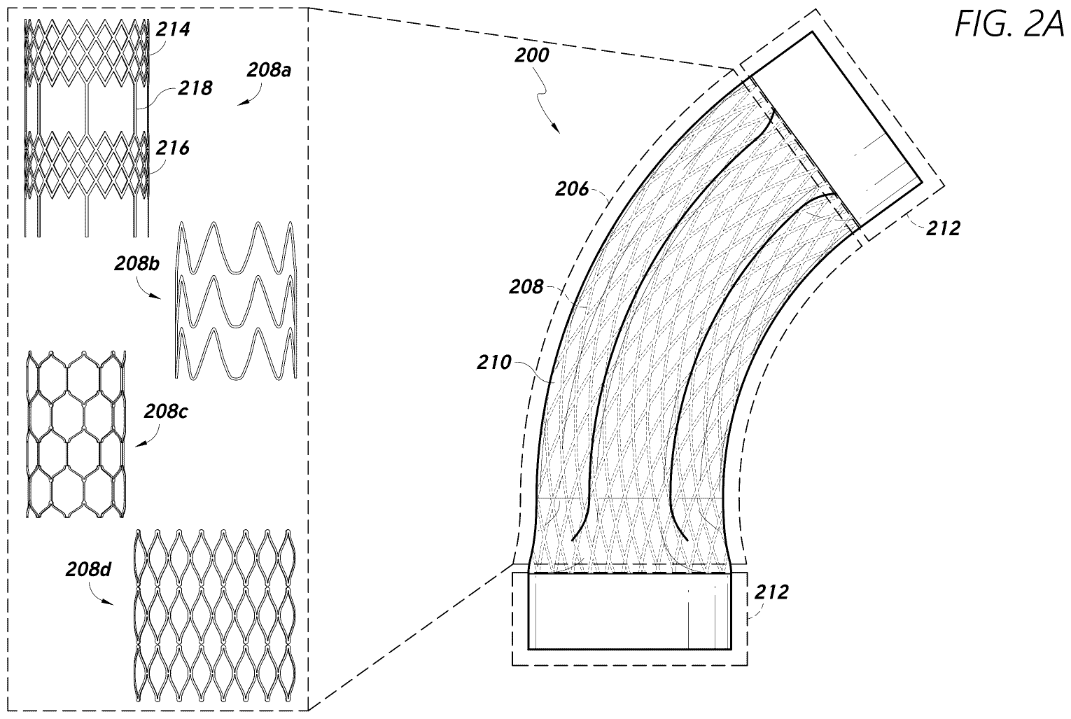

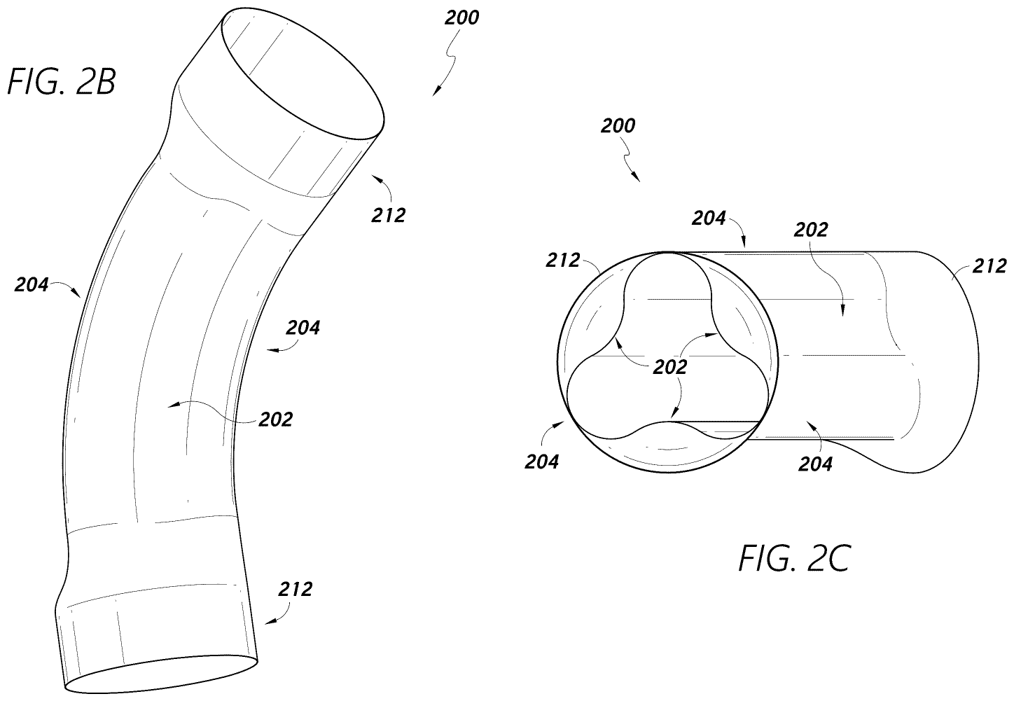

FIGS. 1B-1 and 1B-2 show detailed views of example healthy and unhealthy aortas 120, respectively. The aorta 120 is a compliant arterial blood vessel that buffers and conducts pulsatile left ventricular output and contributes the largest component of total compliance of the arterial tree. The aorta 120 includes the ascending aorta 122, which begins at the opening of the aortic valve 118 in the left ventricle 104 of the heart 100. The ascending aorta 122 and pulmonary trunk 110 twist around each other, causing the aorta 120 to start out posterior to the pulmonary trunk 110, but end by twisting to its right and anterior side. Among the various segments of the aorta 120, the ascending aorta 122 is relatively more frequently affected by aneurysms and dissections, often requiring open heart surgery to be repaired. The transition from ascending aorta 122 to aortic arch 124 is at the pericardial reflection on the aorta. At the root of the ascending aorta 122, the lumen has three small pockets between the cusps of the aortic valve 118 and the wall of the aorta 120, which are called the aortic sinuses or the sinuses of Valsalva. The left aortic sinus contains the origin of the left coronary artery and the right aortic sinus likewise gives rise to the right coronary artery. Together, these two arteries supply the heart with blood.

As mentioned above, the aorta 120 is coupled to the heart 100 via the aortic valve 118, which leads into the ascending aorta 122 and gives rise to the innominate artery 126, the left common carotid artery 128, and the left subclavian artery 130 along the aortic arch 124 before continuing as the descending thoracic aorta 132 and further the abdominal aorta 134. References herein to the aorta can be understood to refer to the ascending aorta 122 (also referred to as the “ascending thoracic aorta”), aortic arch 124, descending or thoracic aorta 132 (also referred to as the “descending thoracic aorta”), abdominal aorta 134, or other arterial blood vessel or portion thereof.

Arteries, such as the aorta 120, can utilize blood vessel compliance (e.g., arterial compliance) to store and release energy through the stretching of blood vessel walls. The term “compliance” can be used herein according to its broad and ordinary meaning, and can refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood can flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta 120, as shown in FIG. 1B-1, runs along a generally straight path, whereas an aged and/or stiffened aorta 120, as shown in FIG. 1B-2, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

As understood by those having ordinary skill in the art, the systolic phase of the cardiac cycle is associated with the pumping phase of the left ventricle, while the diastolic phase of the cardiac cycle is associated with the filling phase of the left ventricle. With proper arterial compliance, a change in volume will generally occur in an artery between high- and low-pressure phases of the cardiac cycle. With respect to the aorta, as blood is pumped into the aorta through the aortic valve, the pressure in the aorta increases and the diameter of at least a portion of the aorta expands. A first portion of the blood entering the aorta during systole may pass through the aorta during the systolic phase, while a second portion (e.g., approximately half of the total blood volume) may be stored in the expanded volume caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance can have a significant effect on perfusion and/or blood pressure in some patients. For example, arteries with relatively higher compliance can be conditioned to more easily deform than lower-compliance arteries under the same pressure conditions. Compliance (C) can be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

A blood vessel that is relatively stiff can experience compliance that is diminished relative to a healthy blood vessel. Due to the stiffness of the blood vessel wall, the blood vessel can expand a relatively limited amount between diastole and systole. That is, during systole, the increased fluid pressure within the blood vessel can result in a relatively small and/or negligible expansion of the diameter of the blood vessel. Due to the limited expansion of the blood vessel, the change in volume in the blood vessel between phases of the cardiac cycle can likewise be limited, and therefore relatively little energy is stored in the blood vessel wall and returned to the blood circulation during low-pressure conditions, resulting in more pulsatile blood flow compared to healthy, compliant tissue.

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

FIG. 1B-2 illustrates the unhealthy aorta 120 with an aneurysm 136, which can include a permanent dilation/enlargement in the blood vessel due to weakened or abnormal tissue. Several sites for an aneurysm can include the Abdominal Aorta (e.g., Abdominal Aortic Aneurysm (AAA)), the ascending aorta, the aortic arch, the descending aorta, the thoracic aorta (e.g., Thoracic Aortic Aneurysm (TAA)), a portion of the aorta that spans several segments (e.g., Thoracoabdominal Aortic Aneurysm (TAAA)), and so on. In some instances, such as asymptomatic progressive aneurysmal dilation, an aneurysm can rupture, causing internal bleeding that presents a serious risk to the patient, such as death.

To treat an aneurysm in a blood vessel, a graft or other medical device can be implanted at the site of the aneurysm. For example, a physician can resect an aneurysmal portion of the aorta and implant a graft thereon. In various solutions, the graft includes a rigid structure, which can increase afterload for the left ventricle, cause long-term detrimental effects to the left ventricle, and/or cause other undesirable consequences.

Although the unhealthy aorta 120 is shown in FIG. 1B-2 with several undesirable characteristics including a more tortuous, curved path (in comparison to a healthy aorta) and an aneurysm 136, the unhealthy aorta 120 can additionally, or alternatively, include other issues/conditions. Further, although many examples are discussed in the context of aneurysms, the devices, methods, and/or systems disclosed herein can be implemented to treat a variety of conditions, such as stiffened blood vessels, acute aortic syndromes (AAS) (including aortic dissection (AD)), penetrating atherosclerotic ulcer (PAU), intramural hematoma (IMH), traumatic aortic injury (TAI), pseudoaneurysm, congenital abnormalities (including the coarctation of the aorta (CoA)), atherosclerotic and inflammatory affections, aortic rupture, genetic diseases (e.g. Marfan syndrome), and so on.

In view of the health complications that can be associated with aneurysms, reduced arterial compliance, and/or other conditions, it can be desirable in certain patients and/or under certain conditions, to treat the affected area and/or at least partially restore/alter compliance properties of the aorta or other blood vessels, or otherwise alter/control flow therein, in order to improve cardiac and/or other organ health.

### Implant Devices

The present disclosure relates to systems, devices, and methods for treating aneurysms and other conditions in blood vessels, such as the aorta or other arterial (or venous) vessel(s), while providing compliance characteristics. Examples of the present disclosure can include a device having a biased cross-sectional shape that includes multiple lobes and multiple deflectable portions between the multiple lobes. The multiple deflectable portions can be configured to expand/deflect radially based on pressure within the fluid vessel (e.g., luminal/radial pressure) to provide a change in volume over the cardiac cycle. For example, the cross-sectional shape of the device can change during expansion and contraction, such as from a first lobular shape having a smaller cross-sectional area to a second more-circular shape having a larger cross-sectional area. Such change in volume can allow the device to mimic compliance of a healthy blood vessel and/or otherwise promote blood flow during, for example, a phase of the cardiac cycle.

The systems, devices, and methods discussed herein can increase blood perfusion/flow and/or restore/provide compliance to the fluid vessels and/or other organs. For example, the compliant-enhancing devices can change form/shape and store energy during higher-pressure periods of the cardiac cycle (e.g., during the systolic phase/period) and deflect radially inward during lower-pressure periods (e.g., during the diastolic phase/period) to return the stored energy to the circulation and increase flow through the vessel. As such, the devices can improve diastolic flow. Further, the systems, devices, and methods of this disclosure can avoid/minimize many of the negative effects that are commonly associated with implanting a medical device in a blood vessel. For example, the compliant-enhancing devices can mimic the expansion and contraction of a healthy blood vessel during phases of the cardiac cycle, which can avoid/minimize afterload to the heart (e.g., reduce left ventricle afterload), in comparison to other solutions that include relatively rigid structures. Moreover, the systems, devices, and methods can maintain a continuous flow pattern into the microvasculature of end-organs (e.g., minimize/avoid pulsatile flow), which can prevent end-organ damage. For example, cerebral, renal, coronary, etc. circulation can be improved.

The devices discussed herein can be implanted at an aneurysmal site or other target site using a variety of approaches, such as a surgical approach (e.g., open surgery) (which may resect a portion of the unhealthily blood vessel) and/or endovascular/minimally invasive approach. When implanted, the device can repair the aneurysmal site and/or otherwise provide an alternative/corrective blood channel through/around the aneurysm or other target site to improve perfusion of the blood through the vessel and/or other organ(s) of the body.

Methods and/or structures disclosed herein for treating a patient also encompass analogous methods and structures performed on or placed on a simulated patient, which is useful, for example, for training, demonstration, procedure and/or device development, and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof, synthetic, or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

Implant devices, methods, and concepts disclosed herein can be described in the context of the aorta. However, such devices, methods, and/or concepts can be applicable in connection with any other artery or blood vessel.

FIGS. 2A, 2B, and 2C illustrate side, side-perspective, and top-perspective views, respectively, of an example device/system 200 (sometimes referred to as the “implant device 200”) configured to be implanted/disposed at a target site and provide compliant characteristics to a fluid vessel. For ease of illustration, the device 200 is illustrated in FIG. 2A with a frame structure for a least a portion of the device 200, whereas FIGS. 2B and 2C illustrate the device 200 without the frame structure. As discussed below, any portion of the device 200 can include or not include a frame, depending on the specific example/design.

The implant device 200 can generally include a tubular form that provides a fluid/blood channel/conduit/lumen to replace/substitute/support a native channel through a fluid/blood vessel. The implant device 200 can be configured/designed to have a biased/default form/shape that includes multiple first sections/portions/areas/segments/walls/wall portions 202 that deflect radially inward and multiple second sections/portions/areas/segments/walls/wall portions 204 positioned/disposed between the first sections 202. FIGS. 2A-2C generally illustrate the device 200 in a default/non-expanded state; however, such state can be a non-default state in some case. In various examples, in a default/non-expanded, the first sections 202 form concaved/recessed/indents/depressions/invaginated portions that generally bend/curve/deflect inwards, and the second sections 204 form lobes/protrusions/projections/convex portions that generally bend/curve/deflect radially outwards. For ease of discussion, the first sections 202 will often be referred to as deflectable portions/sections/areas 202, while the second sections/portions 204 will be referred to as lobes 204. The deflectable portions 202 can generally be configured to expand/deflect radially outward as luminal/radial pressure increases and return/contract to a biased/default state as luminal/radial pressure decreases. This causes the device 200 to exhibit compliant characteristics, similar to a blood vessel.

In various examples, such as that shown in FIGS. 2A-2C, the deflectable portions 202 and lobes 204 extend longitudinally from one end/end portion to another end/end portion of the device 200. The deflectable portions 202 and lobes 204 are shown as extending within a segment/section/area 206 of the device 200 (also referred to as the “compliant/expandable/contractible/elastic segment 206”), which can be a central section of the device 200. The compliant segment 206 can have any length, such as that shown in FIGS. 2A-2C or another shorter or longer length. In other examples, the deflectable portions 202 and/or lobes 204 can be disposed/extend circumferentially (e.g., around the circumference) and/or in other manners. In some examples, the compliant segment 206 includes a feature(s)/area/point between an adjacent deflectable portion 202 and lobe 204, such as a crease, indentation, ridge, scoring, etc., to provide a transition/deflection/deformable location/area, wherein the deflectable portions 202 can at least partially deflect along the feature. Although the device 200 is shown in many examples with three deflectable portions 202 and three lobes 204, the device 200 can include any number of deflectable portions/lobes.

The compliant segment 206 can include a frame(s)/frame structure(s) 208 and/or a covering(s)/cover(s) 210 disposed on the frame 208. A wall of the frame 208 can be a single, circumferentially-wrapped wall, or can be multiple walls, or wall segments. The covering 210 can be disposed around and/or within the inside of the frame 208 (also referred to as “the tubular frame structure 208”), such that the covering 210 generally contacts and conforms to the shape/form of the frame 208 (e.g., expands and contracts along with the frame 208). The covering 210 can be elastic (or include certain elasticity characteristics/properties) to allow the covering 210 to expand and contract with the frame 208. Although various figures illustrate the covering 210 as being disposed around the frame 208, the covering 210 can be disposed within the frame 208 (i.e., the covering 210 is internal relative to the frame 208). The covering 210 can cover one or more internal and/or external portions/surfaces of the frame 208. For instance, the covering 210 can include a fabric/material with different layers, wherein the frame 208 can be embedded between different layers of the fabric/material.

The covering 210 (and/or covering/material of anchoring features 212 or other coverings discussed herein) can be formed of a material/cloth that is able to withstand many cycles without tearing/rupturing or otherwise being damaged. In implementations, the covering 210 (and/or covering/material of the anchoring features 212) comprises a cloth or polymer sleeve which may be at least partially elastic, or alternatively, nonelastic. The covering 210 (and/or covering/material of the anchoring features 212) can be applied over or within the frame in any suitable or desirable manner. For example, the covering 210 (and/or covering/material of the anchoring features 212) can be applied using an electrical or mechanical spinning (e.g., rotary jet spinning, electrospinning, or similar) application process or other deposition process. In some cases, the covering 210 (and/or covering/material of the anchoring features 212) can promote tissue ingrowth within a native blood vessel.

The covering 210, as with a covering/material of the anchoring features 212 or any other covering disclosed herein, can comprise any suitable or desirable material/biocompatible material. For example, a covering can comprise expanded polytetrafluoroethylene (ePTFE), PTFE, thermoplastic polyurethane (TPU), polyester, polyurethane, fluoropolymers (e.g., perfluoroelastomers and the like), polytetrafluoroethylene, polyethylene terephthalate (Dacron), silicones, urethanes, ultra-high molecular weight polyethylene, aramid fibers, and combinations thereof. In some implementations, a covering comprises fabric configured to induce and/or encourage tissue ingrowth with the covering layer(s), or alternatively designed to impede tissue-ingrowth with respect to an area or region of the stent for which endothelialization is not desirable.

In examples, a covering can be configured to promote hemostasis sealing between the device 200 and the blood vessel in which they are implanted. Such sealing may occur at least at the attachment features 212. Coverings described herein can comprise textiles or other materials configured to promote endothelialization, which may help to secure the device 200 to the blood vessel, as well as provide sealing functionality to prevent blood from passing on an outer diameter of the device 200.

As noted above, the compliant segment 206 is configured to change in form/shape based in part on fluid pressure associated with the fluid vessel in which the implant device 200 is implanted. The compliant segment 206 (e.g., the frame 208 and/or covering 210) can be biased to a particular shape/form (also referred to as “a biased/default/relaxed state/form” or “primary state/form”), wherein such biased shape/form is associated with less volume/cross-sectional area than an expanded form. As luminal pressure within the device 200 increases, the deflectable portions 202 can expand/deflect radially outward to the expanded shape/form (also referred to as a “secondary state/form”). In contrast, as luminal pressure decreases, the deflectable portions 202 can collapse/deflect radially inward back to the biased shaped/form. In some instances, the compliant segment 206 includes the frame/frame structure 208 to implement a biased/predefined/default shape/form.

In examples, the compliant segment 206 can change a cross-sectional shape/form to facilitate a change in cross-sectional area and volume for the implant device 200. For instance, the compliant segment 206 can change from a lobular shape to a more circular/rounded shape as luminal pressure increases within the compliant segment 206. The lobular shape can include less cross-sectional area than the more circular/rounded shape for the given/fixed perimeter/wall length. By changing from a lobular shape to a more rounded shape, the compliant segment 206 can expand/increase in at least one dimension (e.g., a dimension/distance between a midpoint of a deflectable wall 202 and a longitudinal axis can increase). Further, by changing from a more rounded shape to a more lobular shape, the compliant segment 206 can contract/decrease in at least one dimension (e.g., a dimension/distance between a midpoint of a deflectable wall 202 and a longitudinal axis can decrease). In examples, the compliant segment 206 can expand/deflect to produce a circle cross-sectional shape, which is the maximum area for the given/fixed perimeter/wall length. That is, the greatest area/volume of the device 200 can be present/achieved when the wall(s) of the device 200 forms a circular cross-sectional shape (e.g., a diameter is substantially constant at every angle about the axis of the device 200).

FIG. 2A illustrates various frame types that can be implemented for the frame 208. As shown, frame 208a can be implemented with a first section 214 (e.g., first set of cells) and a second section 216 (e.g., second set of cells) coupled to/together via wires/segments/struts 218. Such configuration can provide at least some flexibility longitudinally to allow the device 200 to curve/bend with the anatomy. Although two sections are shown, any number of sections can be implemented. Further, as also shown in FIG. 2A, frame sections 208b can be implemented that are more horizontally disposed (which can include frame sections that are independent/disjoint from each other and coupled via a covering and/or struts/wires). Moreover, frames 208c, 208d can be implemented that have a plurality of cells. The frames 208a (sections 214 and/or 216), frame 208c, and/or frame 208d can have a structure comprising a plurality of struts forming an array of cells. Any of the frames 208a, 208b, 208c, and/or 208d can be formed of nitinol or another shape-memory metal or material. In some examples, the frame 208a and/or frame 208b (and/or any of the other frames) can be configured to be cut to a particular size, such as by cutting in between adjacent strut/frame structures/pieces. In some examples, the frame 208 is configured to prevent kinking. Further, in examples, the frame 208 is configured to flex/bend/curve longitudinally, such as to provide some movement/flex with the native vessel. For instance, the frame 208a and/or frame 208b (and/or any of the other frames) can be configured to provide flexibility of the device 200 (e.g., longitudinal flexibility), due to spacing between adjacent struts/frame elements and/or minimal frame structure (e.g., the less structure for the wires 218 of the frame 208a in comparison to the first and second sections 214 and 216, the spacing between adjacent frame structures of the frame 208b (which can be covered with a covering), etc.).

In some examples, the device 200 includes a bare-frame (e.g., bare metal), wherein the frame 208 is not covered internally or externally by a fluid-tight covering. Here, the device 200 can be configured to attach to the native tissue, such that the native tissue can be shaped and/or move along with the frame 208. For example, the natural vessel walls can expand and contract along with the frame 208 based on luminal pressure within the vessel. As such, the vessel walls can be reshaped to the form/shape of the frame 208.

In some cases, the device 200 includes attachment/anchoring/anchor features/sections/segments/structures 212 configured to couple/attach to target anatomy (not illustrated). The attachment features 212 can be coupled to or integral with the compliant segment 206. That is, the attachment features 212 can be separate components/structures that are coupled to the compliant segment 206 or can be integral with the compliant segment 206 (e.g., the compliant segment 206 includes attachment locations/sections). In some cases, the attachment features 212 include specific structure/features/characteristics to attach/couple to the target anatomy, as discussed in further detail below.

Although two attachment features 212 are illustrated, any number of attachment features 212 can be implemented. In some cases, the compliant segment 206 extends between two opposing attachment features 212, such as that shown in FIGS. 2A-2C. In other cases, the compliant segment 206 extends from one end of the device 200 with an attachment feature to another end without an attachment feature. In yet other cases, the device 200 is implemented without specific attachment features and the compliant segment 206 is coupled to the target anatomy. Further, the device 200 (e.g., the compliant segment 206) can coupled to other devices for attachment to the target anatomy.

The compliant segment 206 and/or the attachment segment(s) 212 can include or be referred to as a tubular structure or tubular frame/frame structure (when the compliant segment 206 and/or the attachment segment(s) 212 include a frame). A tubular structure can generally include a lumen, such as to permit fluid/blood flow therethrough. A tubular structure can take a variety of forms/shapes, such as a variety of cross-sectional shapes.

In examples, the attachment features 212 include cross-sectional shapes/forms that are similar to or match a shape/form of the target anatomy. For example, the attachment features 212 can include circular/circle cross-sectional shapes to match the shape/form of a blood vessel, which can facilitate a fluid tight seal with the blood vessel. The device 200 can transition from the lobe-shaped cross section of the compliant segment 206 to the circle cross section of the attachment features 212, with one or more smooth or abrupt surfaces. A segment/section/area between the compliant segment 206 and the attachment features 212 can be referred to as a transition section/segment/area. However, the attachment features 212 can include other forms/shapes. Further, the attachment features 212 can include the same or different lengths, diameters, or other dimensions. In some instances, the compliant segment 206 and/or the attachment features 212 include a barb(s), patch(es), pin(s), coil(s), screw(s), tab(s), hook(s), wire(s), spike(s), or another tissue anchor means configured to embed in and/or hold to the anatomy. In some cases where the compliant segment 206 is coupled/anchored to the anatomy, the anatomy may move inwards and outwards along with the deflectable portions 202.

The attachment features 212 can be coupled to or integral with the covering 210 to form a fluid tight seal with the compliant segment 206 and provide a conduit for fluid flow through the implant device 200. In some cases, the attachment features 212 and covering 210 are separate components that are coupled together. In other cases, the attachment features 212 (or materials/coverings for the attachment features 212) are integral with the covering 210 to form a continuous piece (e.g., the covering 210 extends from one end of the device 200 to the other end of the device 200 through the attachment features 212).

In examples, such as that shown in FIGS. 2A-2C, the attachment segment(s) 212 is formed of a fabric, cloth, or other material. In some cases, the fabric/cloth/material can have less than (or more than, in some cases) a threshold amount of elasticity/expandability. In some examples, the attachment segment(s) 212 are formed of expanded polytetrafluoroethylene (ePTFE), PTFE, thermoplastic polyurethane (TPU), polyester, polyurethane, fluoropolymers (e.g., perfluoroelastomers and the like), polytetrafluoroethylene, polyethylene terephthalate (Dacron), silicones, urethanes, ultra-high molecular weight polyethylene, aramid fibers, and combinations thereof.

The attachment segment(s) 212 can be formed of a material that can be cut to adjust a size/length of the device 200 for the particular application. The attachment segment(s) 212 can be formed of a material that minimizes fraying. In some cases, the attachment segment(s) 212 is implemented with a fabric/cloth/material (which may have less than a threshold amount of elasticity, such as a non-compliant structure/material) when the device 200 is configured/designed for a surgical delivery, wherein the attachment segment(s) 212 are sutured/stapled or otherwise attached to the target anatomy (which can be resected anatomy).

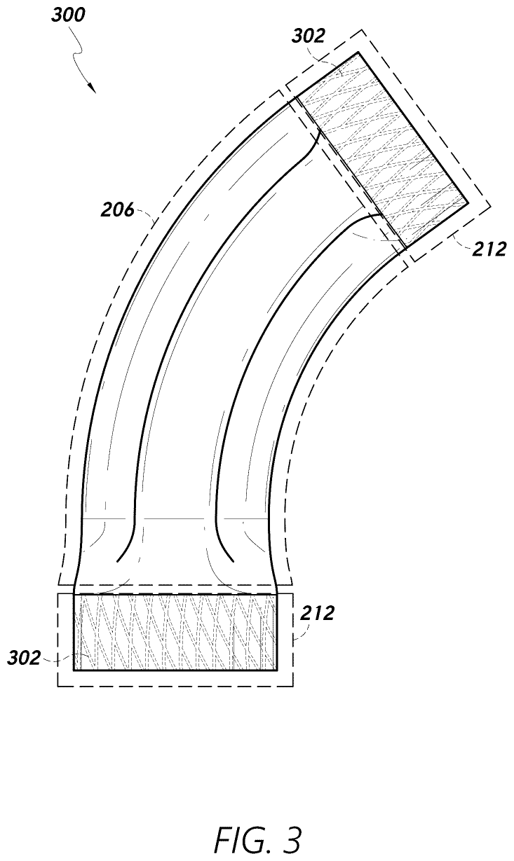

Further, in examples, such as that shown in FIG. 3, the attachment segment(s) 212 includes a frame 302 or other structure configured to be compressed, such as to couple to a delivery system, and/or configured to expand, such as to attach the device 200 to a fluid vessel. For instance, the device 200 can be configured for an endovascular delivery, wherein the attachment segment(s) 212 (e.g., the frame 302) is biased to a particular shape/form (also referred to as “a biased/expanded state/form” or “primary state/form”). The attachment segment(s) 212 can be compressed/collapsed to a compressed/collapsed/delivery state/form (“secondary state/form”) and loaded/disposed/positioned on a delivery system. During delivery, the attachment segment(s) 212 can expand back to or towards the biased/primary state, wherein the attachment segment(s) 212 can apply a force/pressure to the native tissue to hold/anchor the device 200 in place. In some cases, once deployed, compliant characteristics of the device 200 are generally exhibited by the compliant segment 206 and the attachment segment(s) 212 form a more rigid structure (e.g., the luminal pressure may not be sufficient to cause expansion of the attachment segment(s) 212). The frame 302 can be the same as or similar to any of the frames 208a-208d and/or other frames discussed herein. In some cases, the attachment segment(s) 212 is self-expandable, while in other cases a device/dilator is used to expand the attachment segment(s) 212. In some examples where the attachment segment(s) 212 is implemented with the frame 302, the frame 208 of the compliant segment 206 can be coupled to or integral with the frame 302 of the attachment segment(s) 212. However, the frames 208 and 302 can be separate and/or not coupled together. The frames 208 and 302 can include the same covering or different coverings.

The frame 208 of the compliant segment 206, the frame 302 of the attachment segment(s) 212, and/or any other frame can be made of any at least partially rigid material, such as metal, plastic, etc. A frame can be also referred to as a “stent,” “stent frame,” “wire frame,” or “frame structure.” In some examples, a frame can comprise stainless steel, nitinol, etc. In examples, a frame can include shape memory/super elasticity to implement a biased/default form/shape. For instance, a frame can be formed of nitinol or another shape-memory metal or material, which can allow the frame to expand/collapse and return to a biased form. A frame can be formed using any suitable process, such as by stamping or machining the frame structure from a sheet or tube of metal/material. A frame can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and/or rows in the axial, or lengthwise, direction. In some implementations, the array of struts is formed from a sheet of metal, which is rolled into a cylinder to form a tubular/cylindrical form. In examples, a frame can be configured to experience tissue in-growth in one or more areas thereof.

The terms “shape memory,” “shape memory effect,” “shape memory characteristic,” and the like are used herein according to their broad and ordinary meanings, and can refer to, for example, any tendency of a material, once deformed, remodeled, adjusted, or otherwise manipulated or configured from an original and/or set/biased shaped thereof, to return to the original/set/biased shape, form, or structure when a deforming force is removed or reduced. For example, in some contexts, shape memory or the like can relate or refer to the ability of a material/element to deform at a temperature when an external force is applied, maintain the deformed shaped when the external force is removed, and return to the undeformed shape when the element is heated above a particular temperature. Further, the terms recited above can connote, indicate, and/or refer to superelasticity characteristics of a referenced material/element, wherein such shape-memory and/or superelasticity characteristics can relate to the tendency and/or ability of the subject material/element to deform when an external force is applied and return to the undeformed shape when the force is removed. As used herein, a material/element that includes shape memory can be understood to refer the shape memory effect and/or superelasticity. For example, a material/element that includes shape memory properties/characteristics (also referred to as “a shape memory element”) is configured to undergo deformation due to an external force/stress and return to its undeformed shape upon removal of the external force/stress, in some cases by changing the temperature of the material/element, and in other cases without changing the temperature of the material/element. To illustrate, a device with shape memory can include a biased/default shape/form, wherein the device is configured to be compressed, expanded, or otherwise deform when an external force is applied and configured to return to the biased/default shape/form when the external force is removed.

The device 200 can be designed/configured with various shapes to conform/match to the shape/form of the anatomy where the device 200 will be implanted. In one example, as shown in FIGS. 2A-2C and 3, the device 200 can at least partially curve relative to a longitudinal axis of the device 200 for implantation at a site that has some curve, such as the ascending aorta, aortic arch, etc. In another example, the device 200 can form a relatively straight structure with respect to a longitudinal axis of the device 200 for implantation at a portion of a fluid vessel that is relatively straight, such as the thoracic aorta, abdominal aorta, etc. In some cases, the device 200 is configured to bend to conform to a desired shape. The implant device 200 can include a structure with a lumen to provide a path for blood to flow through the implant device 200, wherein such path can be straight or curved.

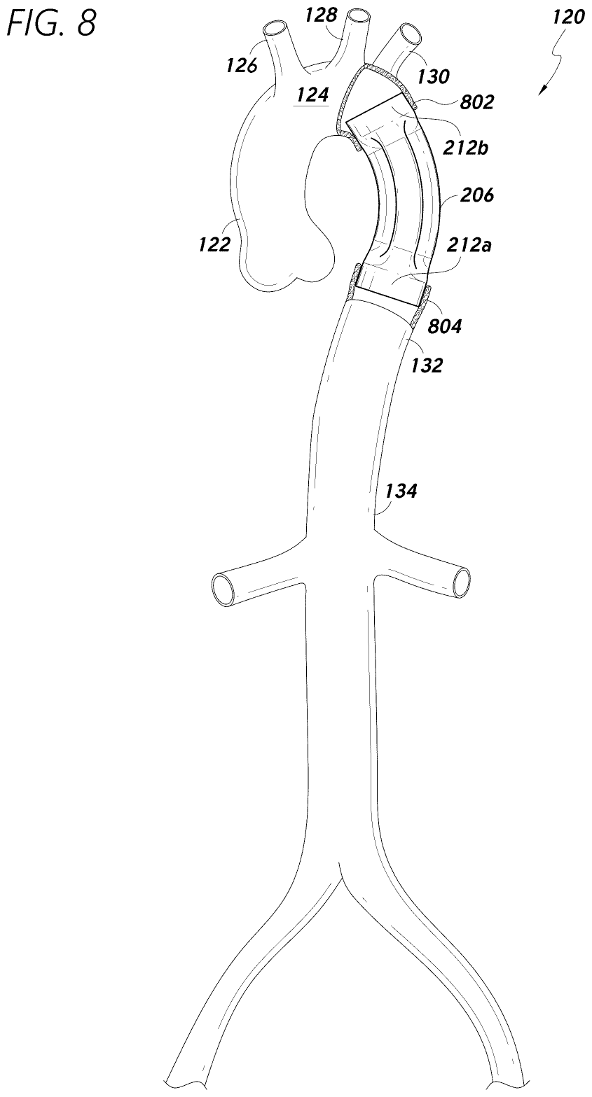

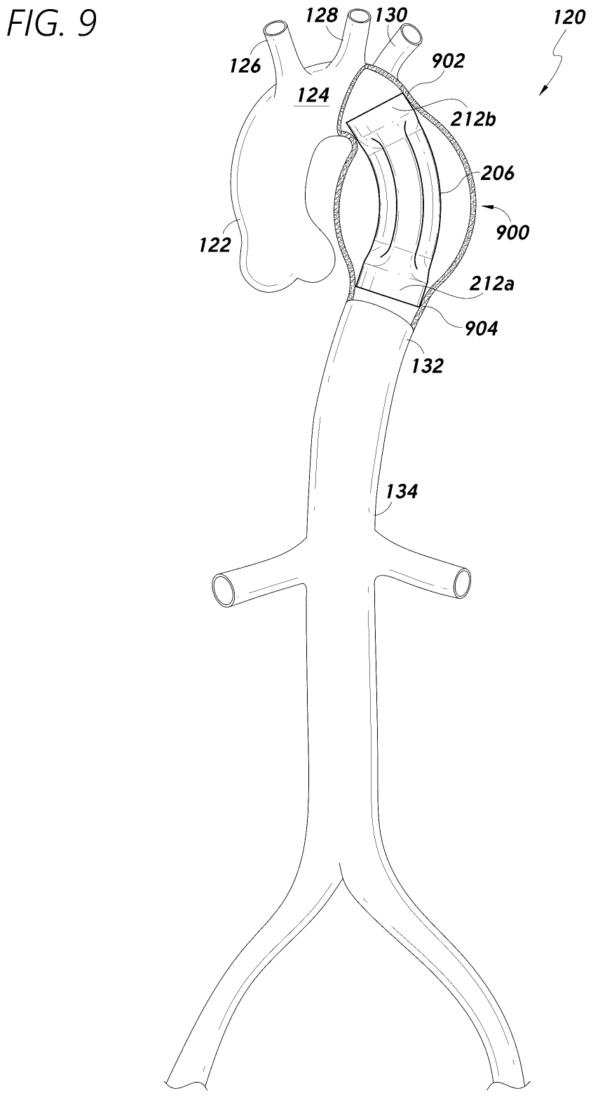

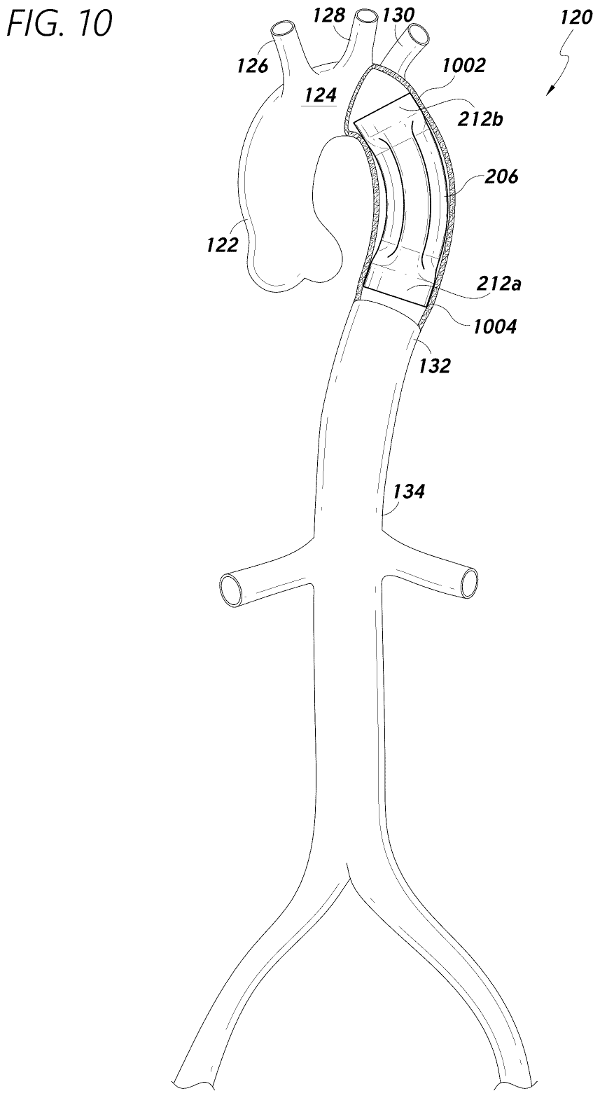

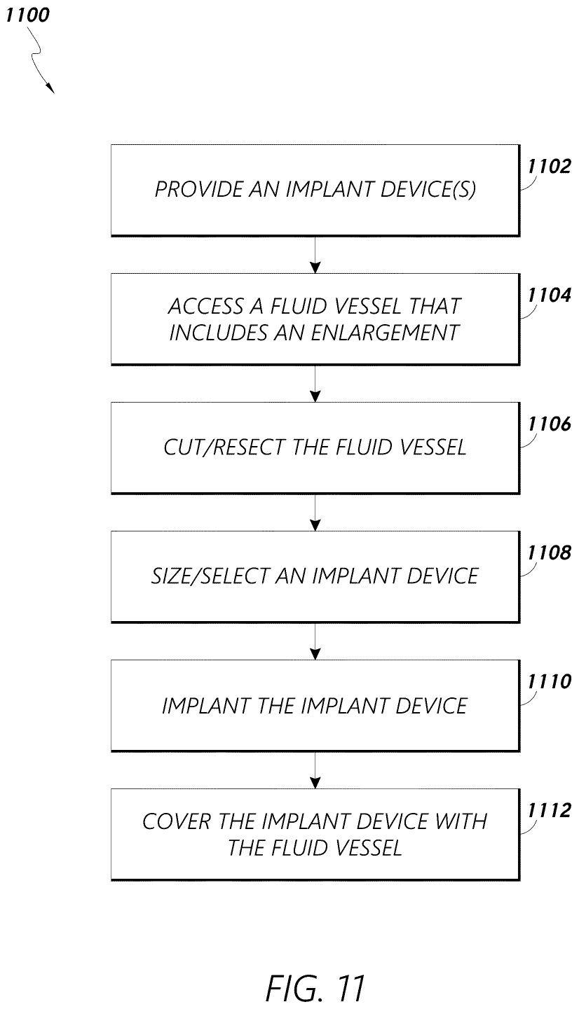

The device 200 can be implanted at a target site of a blood/fluid vessel to treat an aneurysm or other condition and/or to otherwise enhance compliant characteristics of the blood/fluid vessel. In some cases, the device 200 is implanted to replace a section of a blood vessel that has been resected/cut. To illustrate, the device 200 can be implemented as a graft configured to replace an aneurysmal portion of the aorta that has been resected/cut and/or removed, such as that shown in FIG. 8. In other cases, the device 200 is implanted within a blood vessel while maintaining the tissue of the blood vessel, even if such tissue is in an undesirable state. To illustrate, the device 200 can be implemented as a stent at an aneurysmal site without resecting/cutting the aneurysm, wherein the device 200 can expand and contract within the space of the aneurysm, such as that shown in FIG. 9. In yet other cases, the device 200 is implanted within a fluid vessel without an aneurysm and/or in other contexts to enhance compliant characteristics of the fluid vessel, such as that shown in FIG. 10.

In examples, the term graft is used in its broad and ordinary meaning and can refer to a device that is used to replace a portion of native tissue that is removed, cut, etc. Although a graft is generally implanted through a surgical procedure, a graft can be implanted through an endovascular procedure (e.g., with a catheter/delivery system). Further, in examples, the term stent is used in its broad and ordinary meaning and can refer to a device that includes a frame-like structure and/or is configured to support tissue in a native state. A stent can generally be configured to compress for delivery to a target site and expand to couple/anchor the device to native tissue. Although a stent is generally implanted though an endovascular procedure, a stent can be implanted in a surgical procedure. In some cases, a graft includes a characteristic(s) of a stent and/or a stent includes a characteristic(s) of a graft. As discussed herein, the device 200 can include properties/characteristics of a graft, a stent, and/or other structures. For instance, the device 200 can be implanted through a surgical approach and/or an endovascular approach. In examples, the device 200 is implemented as a stent-graft that includes a least one characteristic of both a stent and a graft.

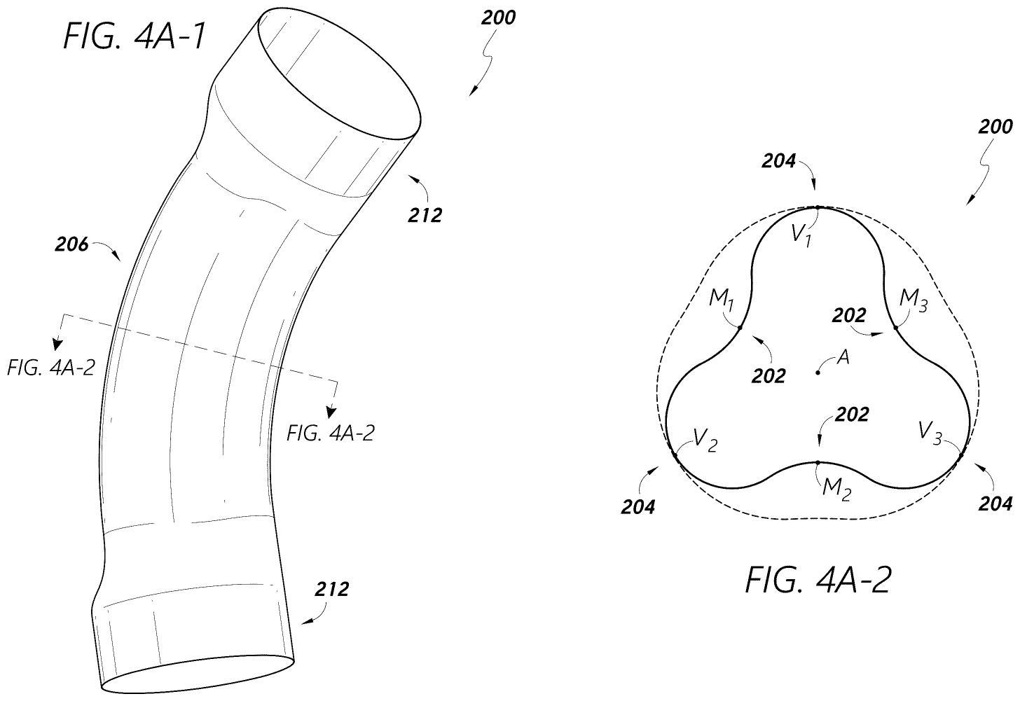

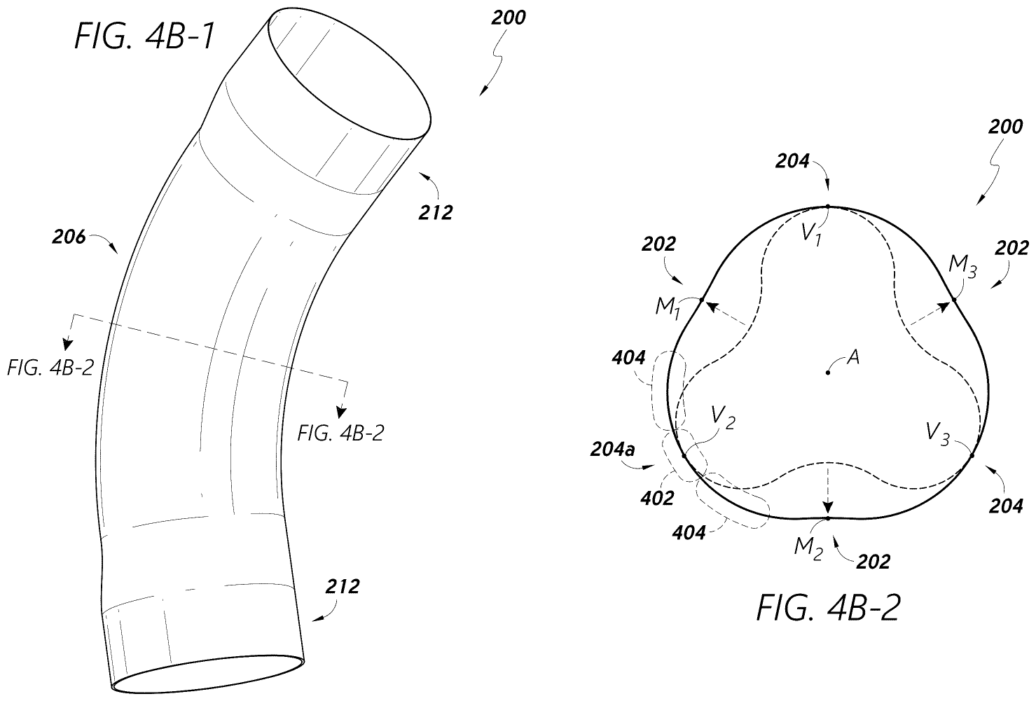

FIGS. 4A-1 and 4A-2 illustrate an example of the device 200 in a relaxed/default/non-expanded/unpressurized state/form (also referred to as a “diastolic configuration/state”), wherein FIG. 4A-2 shows a cross-sectional view through the compliant segment 206. Here, sidewalls/walls of the lobes 204 can be outwardly-curved (e.g., bowed/deflected outward) with respect to an axis A of the device 200 such that the sidewalls are concave from the perspective of the axis A and convex from the perspective of the exterior of the device 200, wherein each of the sidewalls forms a vertex/apex V (represented with V₁, V₂, and V₃). An individual vertex V is generally the farthest point on an individual lobe 204 from the axis A. Further, sidewalls/walls of the deflectable portions 202 can be inwardly-curved (e.g., bowed/deflected inward) with respect to an axis A of the device 200 such that the sidewalls are convex from the perspective of the axis A and concave from the perspective of the exterior of the device 200. A midpoint M of each sidewall (represented with M₁, M₂, and M₃) can be a point/portion of the sidewall that is closest to the axis A.

Descriptions of a device in a relaxed/default/non-expanded/unpressurized configuration/state/form can relate to a configuration that a device naturally assumes in the absence of tension on the device wall(s) from external forces (e.g., ambient fluid pressure, physical contact forces, etc.). For example, the device 200 can include a non-expanded configuration during diastole when luminal pressure is smaller. In the default state, the device 200 includes a lobe-shaped cross-sectional shape, producing a first, smaller cross-sectional area/volume, in comparison to the expanded state discussed in reference to FIGS. 4B-1 and 4B-2.

Luminal pressure forces against the device 200 wall can increase the hoop stress on the device 200, which may force the device 200 to assume a more-circular shape, as shown in FIGS. 4B-1 and 4B-2. The resulting hoop stress, also referred to as “tangential stress” or “circumferential stress,” from luminal pressure increase a radially-outward force along the device's 200 inner circumference, such stresses/forces being tensile in nature, which can tend to cause the device 200 to expand (e.g., the deflectable portions 202 to deflect radially outward). For example, as blood pressure increases, the hoop stress on the walls of the device 200 may force the deflectable portions 202 to deflected radially outward towards a more circular shape.

FIGS. 4B-1 and 4B-2 illustrate an example of the device in a non-relaxed/expanded state/form (also referred to as a “systolic configuration/state”), with FIG. 4B-2 showing a cross-sectional view through the compliant segment 206. Here, sidewalls/walls of the lobes 204 can be more flattened, wherein the sidewalls can still be somewhat concave from the perspective of the axis A and convex from the perspective of the exterior of the device 200. The vertex V can remain in substantially the same position, even though the vertex V may move away from the axis A by some amount (e.g., less than a threshold). Further, sidewalls/walls of the deflectable portions 202 can be outwardly-deflected (and/or outwardly bowed/curved in some cases) with respect to an axis A of the device 200 such that the midpoint(s) M is moved farther from the axis A (e.g., a distance between a respective midpoint M and the axis A increases). As shown, the deflectable portions 202 are deflected radially outward to form a more-circular boundary/outer perimeter cross-sectional shape. The deflectable portions 202 can flatten out, as shown in FIGS. 4B-1 and 4B-2. In some cases, the deflectable portions 202 can deflect even more radially outward to a more bowed shape (e.g., further increase a distance between the respective midpoint M and the axis A).

Descriptions of a device in a non-relaxed/expanded/pressurized configuration/state/form can relate to a configuration that a device assumes with tension on the device wall(s) from external forces (e.g., ambient fluid pressure, physical contact forces, etc.). For example, the device 200 can include an expanded configuration during systole when luminal pressure is larger. In the expanded state, the device 200 includes a more circular cross-sectional shape, producing a second, larger cross-sectional area/volume, in comparison to the non-expanded state.

As noted above, in examples the deflectable portions 202 are configured to deflect radially outward while at least a portion of the lobes 204 remains relatively stationary/fixed or deflect by a minimal amount. For instance, as shown in FIG. 4B-2, a vertex/apex section/portion 402 of the lobe 204a can deflect relatively little (less than a threshold or in comparison to sections 404). Further, the sections 404 of the lobe 204a next to the apex section 402 can deflect more than the threshold or the apex section 402. However, in some cases, the apex section 402 can deflect/expand/move radially outward by some amount, which may increase a distance between adjacent lobes 204. In any event, the respective midpoint M for a given deflectable portion 202 can generally deflect/move by an even larger amount than the sections 402 and/or 404. In examples, the greatest amount of deflection can generally occur at the respective midpoint M for a given deflectable portion 202 and the least amount of deflection can occur at the respective vertex V of the lobe 204, wherein the amount of deflection can decrease from the midpoint M to the vertex V.

In FIG. 4B-2, the lengths of the sections 402 and 404 of the lobe 204a are provided for illustrative purposes. That is, one or more sections of a lobe 204 can be longer or shorter in length, such that the section of wall that is considered a lobe 204 (or a specific portion of a lobe) and the section of wall that is considered a deflectable portion 202 can vary in length/dimension.

The transition of the device 200 from a more lobular shape (as shown in FIGS. 4A-I and 4A-2) to the more-circular shape (as shown in FIGS. 4B-1 and 4B-2) causes energy to be stored in the device 200 (e.g., in the elasticity and/or shape memory thereof), such that energy is returned to a blood vessel in which the device 200 is implanted, and therefore to the blood circulation, when the device 200 transitions back to the more lobular shape as pressure decreases. With respect to implantation within the aorta or other arterial blood vessel, the systolic phase of the cardiac cycle, during which pressure levels in the aorta/arteries are relatively higher, causes the expansion of the device 200 to the more-circular shape (as shown in FIGS. 4B-1 and 4B-2). The more circular shape may provide a larger area/volume within the device 200, wherein a circle cross-sectional shape can produce the largest/maximum area/volume. Therefore, any deviation from a circular/cylindrical form can decrease the area/volume within the device 200. In the diastolic phase, which is associated with relatively lower arterial blood pressure levels, the device 200 assumes a more lobular shape (as shown in FIGS. 4A-1 and 4A-2).

In some examples, the compliant segment 206 is configured to change in shape/form from a non-expanded state to an expanded state (or vice versa) without a change in (or with less than a threshold amount of change to) a perimeter/outer wall length. For instance, the deflectable portions 202 can be configured to change from an inward deflected position to an outward deflected position with minimal change to a length of a cross-sectional perimeter of the compliant segment 206.

In some examples, the device 200 can provide relatively large volume changes, in comparison to a native fluid vessel in a healthy state. For example, in a non-expanded/contracted/default state, the deflectable portions 202 can be deflected radially inward, such that the cross-sectional shape of the compliant segment 206 is smaller than a cross-sectional area of a native blood vessel when contracted (or expanded). Further, in an expanded state, the deflectable portions 202 can expand/deflect radially outward such that the cross-sectional area is larger than the cross-sectional area of the native blood vessel when expanded, in some cases by more than a threshold amount.

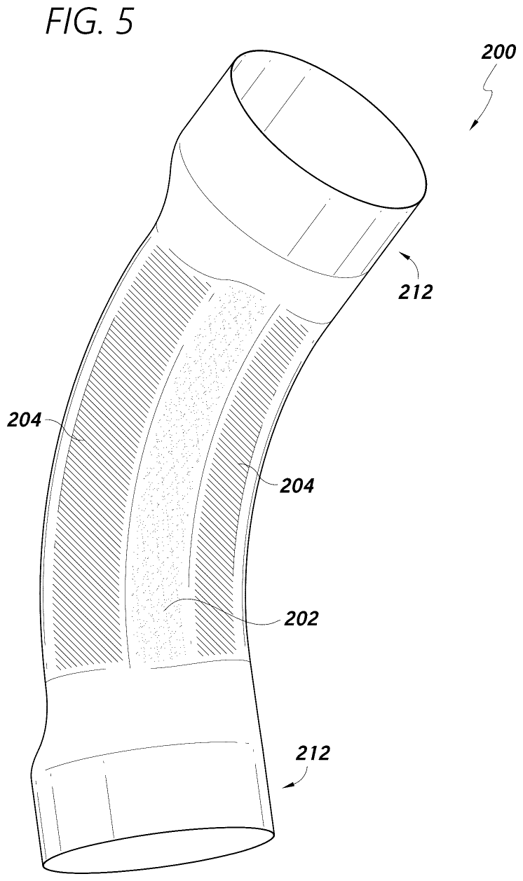

FIG. 5 illustrates an example of the device 200 implemented/configured with different properties/characteristics for the deflectable portions 202 and lobes 204. For example, the deflectable portions 202 and the lobes 204 can include or be formed of different frame patterns/designs/types, materials (e.g., types of materials), etc. In one illustration, the deflectable portions 202 include (are formed of) one or more first frames and the lobes 204 include (are formed of) one or more second frames. The one or more first frames can include/have a first frame pattern/design (which can be based on a density, positioning, attachment, shape, etc. of cells/struts of the one or more first frames), while the one or more second frames can include/have a second frame pattern/design that is different than the first frame pattern/design. In another illustration, the deflectable portions 202 include (are formed of) a first material/material type (e.g., a metal, plastic, steel, nitinol, thermoplastic polyurethane (TPU), rubber, etc.) and the lobes 204 include (are formed of) a second material/material type that is different than the first material (e.g., different types of the same substance or different types of substances). In some instances, different frame patterns/designs and/or materials/material types can exhibit different elasticity, deformation, or other properties, such that the deflectable portions 202 can be more (or less) elastic/deformable than the lobes 204. As such, the deflectable portions 202 can include first characteristics/properties, while the lobes 204 can include second characteristics/properties, in some examples. In some cases, the different characteristics/properties can allow the deflectable portions 202 to deflect more easily than the lobes 204. However, the deflectable portions 202 can be configured to deflect more easily without having different characteristic/properties, which can be due to a biased shape/form of the compliant segment 206, as discussed herein.

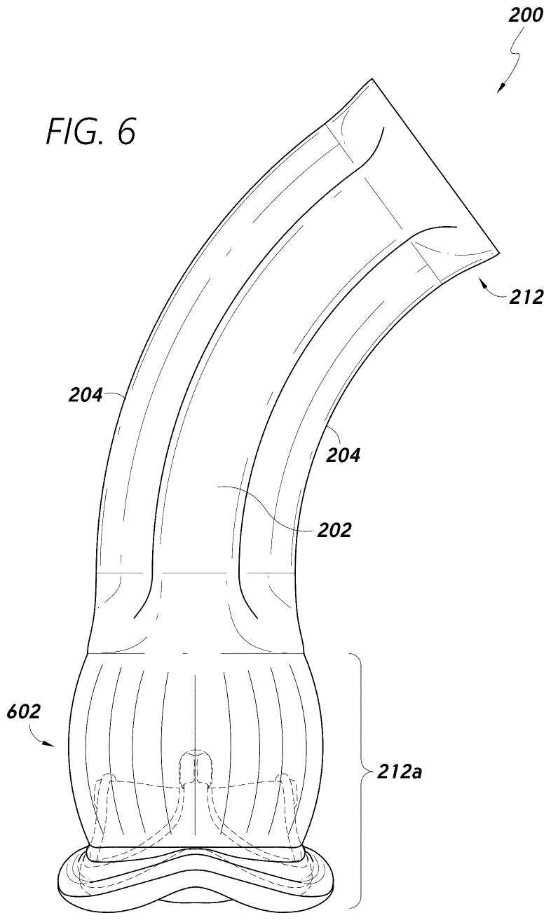

FIG. 6 illustrates the device 200 in an example with a prosthetic valve 602 attached thereto. In particular, the prosthetic valve 602 can be coupled/attached to or replace the attachment segment 212a. The prosthetic valve 602 can be configured to be implanted at a native valve to replace native valve function, while the compliant segment 206 (i.e., the deflectable portions 202 and lobes 204) can provide compliance enhancing functions to the native vessel. In one example, the device 200 of FIG. 6 is implanted with the prosthetic valve 602 positioned at the aortic valve and the rest of the device 200 extending into the ascending aorta and/or aortic arch. As shown, the device 200 can be at least somewhat curved to fit to the ascending aorta. However, the device 200 can be more curved or straight depending on the implantation context. The prosthetic valve 602 can include one or more leaflets, an annulus feature, and/or other features to mimic/replace a native valve.

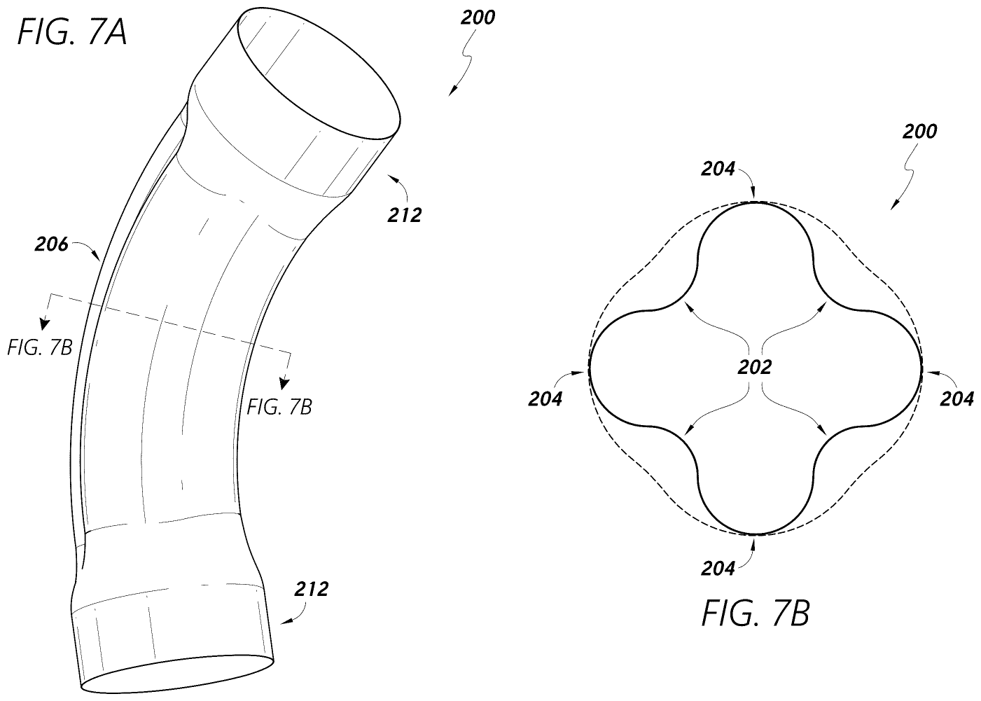

FIGS. 7A and 7B illustrate the device 200 in an example with four deflectable portions 202 and four lobes 204, wherein the device 200 is shown in a default/non-expanded state/form with the deflectable portions 202 deflected radially inward. FIG. 7B shows a cross-sectional view through the compliant segment 206. As shown, the deflectable portions 202 and lobes 204 can be similar to any of the other deflectable portions 202 and lobes 204 discussed herein, respectively, except that four of each element are presented. As noted above, the device 200 can be implemented with any number of deflectable portions 202 and/or lobes 204.

FIG. 8 illustrates the implant device 200 implanted within example anatomy of the patient, namely a resected portion of the aorta 120. Here, the implant device 200 is designed/configured for delivery/implantation through a surgical approach. For example, the attachment segments 212 can be configured to be attached to the native tissue of the aorta with a suture, staple, or another attachment element. In some instances, the attachment segments 212 include a cloth/material to facilitate attachment. The implant device 200 can be curved to match/conform to the shape of the portion of the aorta 120 that the implant device 200 replaces.

As shown, the aorta 120 is resected and the attachment segment 212b is attached/sealed/anchored to an upper/upstream portion 802 of the aorta 120 (e.g., a first location) and the attachment segment 212a is attached/sealed/anchored to a lower/downstream portion 804 of the aorta 120 (e.g., a second location). The aorta 120 can be resected before, during, or after implantation. In some cases, one or more of the attachment segments 212a, 212b are disposed/slid within the aorta 120, as shown in FIG. 8. In other cases, the aorta 120 is disposed within one or more of the attachment segments 212a, 212b. The attachment segments 212a, 212b can be attached to the aorta 120 in a variety of manners, such as by using sutures, bands, or other anchoring/attachment structures/means that are configured to provide a fluid tight seal between the device 200 and the aorta 120. The attachment segments 212a, 212b can be similar in diameter and/or cross-sectional shape to the aorta 120 (e.g., circular) so that the attachment segments 212a, 212b can fit within or around the resected aorta 120.

In examples, the device 200 is coupled to native tissue such that the native tissue is positioned/repositioned over a portion of the device 200. For example, a cut portion of the aorta 120 can be folded over one or more portions of the device 200 and/or tissue may grow over the device 200 after implantation. This can provide a barrier to protect the surrounding tissue from contacting the device 200 directly, such as the compliant segment 206 that expands and contracts. However, the device 200 can additionally, or alternatively, include a covering that minimizes tissue friction.

In the example of FIG. 8, the device 200 can be implemented as a graft, wherein an aneurysmal portion of the aorta 120 is resected/removed so that the implant device 200 replaces the aneurysmal portion. The device 200 can be implanted through a surgical procedure or another procedure. In this example, the compliant segment 206 is free to expand and contract without constraint from vessel walls, in contrast to some cases where the compliant segment 206 is restricted by being implanted within the aorta 120. Further, the graft implementation can allow the compliant segment 206 to be designed to provide a relatively large volume change (e.g., more than a threshold, more than a native portion of the aorta of similar length, etc.), thereby maximizing compliance characteristics of the device 200. Moreover, the graft implementation can avoid blood or other fluids from collecting within the aorta 120 between the device 200 and an inner wall of the aorta 120.

Although the device 200 is illustrated as implanted within the aorta 120 in this example, the device 200 can be implanted within other anatomy and/or positioned elsewhere within the aorta 120. Further, the device 200 can be implanted in anatomy that is not resected.

FIGS. 9 and 10 illustrates the implant device 200 implanted within example anatomy of the patient, namely a portion of the aorta 120 that is not resected. Here, the implant device 200 is designed/configured for delivery/implantation through an endovascular approach. In the examples of FIGS. 9 and 10, the implant device 200 is generally implemented as a stent or stent-grant and/or implanted through an endovascular procedure or another minimally invasive procedure, which can avoid complications associated with more invasive surgical procedures. In such minimally invasive procedures, the aorta 120 is generally not resected. However, other types of procedures can be implemented.

As shown in FIG. 9, the device 200 is disposed within the aorta 120 to position the compliant segment 206 within a space created by the aneurysmal portion 900 (i.e., the aneurysmal sac). That is, the implant device 200 is positioned such that the compliant segment 206 is within a dilated/deformed/enlargement portion/space of the aneurysm (i.e., the aneurysmal sac). In such position, the compliant segment 206 is free to expand and contract within the space of the aneurysmal portion 900, which may generally be larger in space relative to neighboring healthy portions of the associated vessel. In view of this positioning, the compliant segment 206 can be designed to provide a relatively large volume change (e.g., more than a threshold, more than a native portion of the aorta of similar length, etc.), thereby maximizing compliance characteristics of the implant device 200 and/or maximizing the space created by the aneurysm.

In the example of FIG. 9, the attachment segment 212b is sealed/attached/anchored to an inner surface/wall of an upper/upstream portion 902 of the aorta 120 above the aneurysm and the attachment segment 212a is sealed/attached/anchored to an inner surface/wall of a lower/downstream portion 904 of the aorta 120 below the aneurysm. This can fluidly seal the device 200 to the aorta 120 to cause blood to flow through a lumen in the implant device 200 and avoid/minimize blood flow around the implant device 200. In some cases, the device 200 provides a sealed path for blood to flow through the implant device 200 and prevent blood from collecting around the implant device 200 between the implant device 200 and the native vessel, which can lead to complications. Although the implant device 200 is illustrated in the example of FIG. 9 as implanted within the aneurysmal portion 900, the implant device 200 can be implanted within other anatomy and/or positioned elsewhere. For example, the implant device 200 can be disposed within other dilated tissue/enlargements, such as other anatomy that has been dilated in the same or other ways, other portions of the aorta 120 that have been dilated, etc.

Meanwhile, in the example of FIG. 10, the implant device 200 is implanted within the aorta 120 that is free of an aneurysm, at least at the location where the device 200 is disposed. Here, the attachment segment 212b is sealed/attached/anchored to an inner surface/wall of an upper/upstream portion 1002 of the aorta 120 and the attachment segment 212a is sealed/attached/anchored to an inner surface/wall of a lower/downstream portion 1004 of the aorta 120. Although the implant device 200 is illustrated in the examples of FIG. 9 as implanted within the aorta 120, the implant device 200 can be implanted within other anatomy and/or positioned elsewhere, such as another fluid vessel to provide compliant characteristics.

In the examples of FIGS. 9 and 10 (or other examples), one or more portions of the implant device 200 (e.g., the attachment segments 212 and/or the compliant segment 206) can be implemented as one or more expandable frames/structures that are self-expandable or device-expandable to radially expand the implant device 200 to an operational state. For example, the device 200 can be configured to be compressed and loaded onto a delivery device/system and expanded during deployment to an operational state. During deployment, the attachment segments 212 can be configured to expand to attach to the native tissue of the aorta 120. When implanted, the attachment segments 212 and/or the compliant segment 206 can seal to tissue/anatomy to provide a fluid path through the implant device 200 and avoid blood flow into a space between the implant device 200 and the inner wall of the aorta 120 (e.g., avoid blood from collecting in the aneurysmal sac). The attachment segments 212 and/or the compliant segment 206 can be similar in cross-sectional shape to the aorta 120 so that the attachment segments 212 and/or the compliant segment 206 can fit within the associate vessel.

In some examples, in an implanted state, the attachment segments 212 (and/or the compliant segment 206, in some cases) can generally include a diameter/cross-sectional dimension that is larger than the inner diameter/cross-sectional dimension of the associated vessel in which the implant device 200 is implanted. For example, the attachment segments 212 (and/or the compliant segment 206) can each, in an expanded form, have some amount of oversizing (e.g., by a particular percentage, value, etc.), such that an outer diameter is larger than or the same as an average or patient specific inner diameter of a healthy/neighboring blood vessel. In an expanded state, the attachment segments 212 (and/or the compliant segment 206) can apply a radial force to anchor the device 200 to the anatomy. However, the attachment segments 212 (and/or the compliant segment 206) can include anchoring features, such as barbs, wires, hooks, etc.

In examples, the implant device 200 is designed to replace a native fluid vessel that includes branches. For instance, the device 200 can include an upper tubular/tube portion and two or more lower tubular/tube portions that divide/split/diverge from the upper tubular portion (e.g., a Y-shaped implant device or upside-down Y-shaped device depending on the orientation). A Y-shaped device can be configured for various bifurcations, such as the aortic bifurcation. Although two branches are discussed, any number of branches can be implemented. An upper and/or lower tubular/tube portion can include a compliant segment (the same as or similar to the compliant segment 206) and/or an attachment feature (the same as or similar to the attachment feature 212).

A branch segment of the implant device 200 can be configured to match the shape/dimension and/or location of the native branch vessels (e.g., relative to the main native vessel). For instance, the implant device 200 can be designed to replace the aortic arch with the branch segments dimensioned/shaped to the dimensions/shape of supra-aortic vessels or other vessels, such as a coronary artery, brachiocephalic artery, common carotid artery, subclavian artery, branch for cardiopulmonary perfusion, etc. However, the implant device 200 can be implemented/designed to replace other native vessels. Further, in examples, the implant device 200 can be cut at one or more ends to configure the implant device 200 for a particular application/anatomy, such as to fit/match a size of a resected/cut portion of a native vessel.

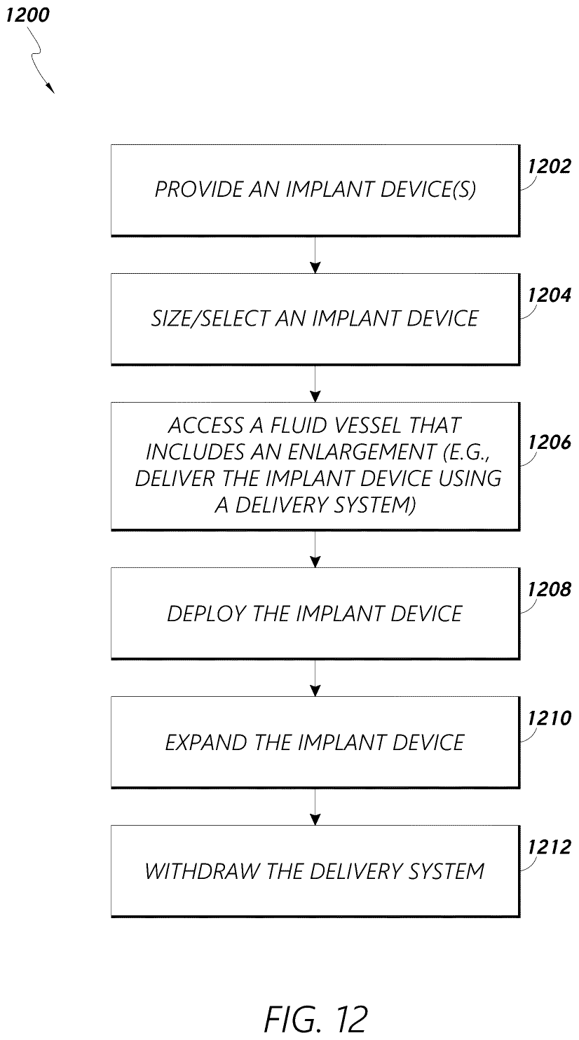

FIGS. 11 and 12 illustrate flow diagrams for process 1100 and 1200, respectively, for implanting an implant device (including any of the implant devices discussed herein) within anatomy in accordance with one or more examples. In examples, the process 1100 of FIG. 11 relates to a more invasive/surgical procedure, wherein the target site is accessed through a surgical approach and/or the anatomy/tissue is surgically cut/resected. In contrast, the process 1200 of FIG. 12 relates to an endovascular/minimally invasive procedure, wherein the target site is accessed through an endovascular/minimally invasive approach and/or the anatomy/tissue is generally not cut/resected. However, any of the blocks/acts discussed for the process 1100 and/or the process 1200 can be implemented in the context of a surgical or endovascular/minimally invasive approach. Further, a block/act illustrated for the process 1100 of FIG. 11 can be implemented for the process 1200 of FIG. 12, or vice versa. Although the blocks are illustrated in a particular order, the order of the blocks can be modified. Further, one or more of the blocks can be eliminated from the processes 1100 and/or 1200.

Although various aspects of the processes 1100 and 1200 and certain other examples are described herein in the context of the aorta, the devices of the present disclosure can be implanted in other arterial or venous blood vessels, such as the inferior vena cava. Further, although the processes 1100 and 1200 and accompanying illustrations are presented with respect to the implantation of a single compliance-enhancement implant device, the processes 1100 and 1200 can involve implanting multiple compliance-enhancement implant devices in various positions within the aorta and/or other fluid vessels.

In FIG. 11, at block 1102, the process 1100 includes providing an implant device(s). The implant device can include any of the implant devices discussed herein, such as the implant device 200. In one non-limiting example, the implant device is implemented as a graft that includes an attachment feature(s) configured for attachment/anchoring to a resected portion of native tissue, such as a cloth or other material configured for suturing to native tissue. In some instances, the attachment feature(s) is configured without (or with) the capability to expand. However, the implant device can be configured in other manners.

At block 1104, the process 1100 includes accessing a fluid vessel that includes an enlargement. For example, a physician/user or robotics system can surgically open a patient to access an aneurysm/target site in the aorta or another fluid vessel. Alternatively, or additionally, in some cases, a catheter/medical tool is implemented to access the target site through a percutaneous access point/port or natural orifice.

At block 1106, the process 1100 includes cutting and/or resecting the fluid vessel. For example, a physician/user or robotics system can cut/resect an aneurysm in the aorta, which can include removing at least a portion of the aneurysmal tissue. In some cases, a catheter/medical tool is implemented to cut/resect the target tissue.

At block 1108, the process 1100 includes sizing and/or selecting the implant device. For example, an aneurysm/aneurysmal portion of the aorta can be measured using various techniques to determine a size/dimension of the aneurysm/aneurysmal portion, such as a length, width, diameter, or other dimension. This can include measuring a distance between resected portions of the fluid vessel (when performed after block 1106) and/or measuring a length or other dimension of the aneurysm/aneurysmal portion before the aneurysm/aneurysmal portion is resected/cut (when performed before block 1106). In examples, a size/dimension of neighboring health tissue is measured/determined, such as a diameter or other dimension of the aorta within a predetermined distance to the aneurysm/aneurysmal portion.

In examples, an implant device can be cut to a length that satisfies/matches the length of the aneurysm and/or resected portion. For instance, one or more ends/attachment features of an implant device can be cut to create the appropriate longitudinal length for the implant device.

Further, in examples, multiple implant devices are available with different sizes/dimensions, so that a physician/user can select the most appropriate implant device for the patient/anatomy. To illustrate, attachment segments and/or compliant segments of different implant devices can include different diameters and/or lengths such that a physician/user can select an implant device that satisfies/matches the size/dimension of the aneurysm/aneurysmal portion and/or the size/dimension of neighboring health tissue. For instance, an implant device that has attachment features with the same/similar diameter as the aorta can be selected.

As such, a measured/determined dimension of anatomy of a patient can be used to determine a device size for an implant device and/or provide/select the appropriate implant device.

At block 1110, the process 1100 includes implanting the implant device. For example, a physician/user or robotic system can attach a first attachment feature of the implant device (at a first end of the device) to a first location above the enlargement/resected portion and attach a second attachment feature of the implant device (at a second end of the device) to a second location below the enlargement/resected portion. The implant device can be attaching using sutures and/or other attachment features/means. The implant device can be implanted before or after the fluid vessel is resected/cut (i.e., before or after block 1106). In some examples, the implant device is implanted at or near a native valve. In some cases where the implant device includes a prosthetic valve, the prosthetic valve can be placed at the native valve.

The term “suture” is used herein according to its broad and ordinary meaning and may refer to any elongate cord, strip, strand, line, rope, wire, filament, tie, string, ribbon, strap, or portion thereof, or other type/form of material used in medical procedures (e.g., ePTFE suture, for example, GORE-TEX® sutures. W. L. Gore, Newark. Delaware). Furthermore, examples of the present disclosure can be implemented in connection with non-surgical and/or non-biological suture/line tensioning. With respect to the present disclosure, one having ordinary skill in the art will understand that a wire or other similar material can be used in place of a suture. Furthermore, in some contexts herein, the terms “cord” and “suture” can be used substantially interchangeably. In addition, use of the singular form of any of the suture-related terms listed above, including the terms “suture” and “cord,” can be used to refer to a single suture/cord, or to a portion thereof. For example, where a suture knot or anchor is deployed on a distal side of a tissue portion, and where two suture portions extend from the knot/anchor on a proximal side of the tissue, either of the suture portions can be referred to as a “suture” or a “cord,” regardless of whether both portions are part of a unitary suture or cord. Anchor guides in accordance with aspects of the present disclosure can be utilized in methods for controlling spacing of surgical sutures. Such sutures and/or associated anchors can be introduced to the target implantation site using a minimally invasive incision and/or can be implanted/deployed while the patient's heart is beating. Furthermore, sutures can be used with a pledget to reduce tissue damage and/or spread the suture load over a broader surface area.

At block 1112, the process 1100 includes covering the implant device with the fluid vessel. For example, a physician/user or robotics system can take a loose piece of the aorta that was resected and is not attached to the implant device and cover at least a portion of the implant device. This can provide a barrier between the implant device and the neighboring tissue of the patient. Although illustrated in FIG. 11, in various examples block 1112 is not performed (i.e., the process 1100 does not include block 1112).

In FIG. 12, at block 1202, the process 1200 includes providing an implant device(s). The implant device can include any of the implant devices discussed herein, such as the implant device 200. In one non-limiting example, the implant device is implemented as a stent or stent-graft that is configured to be compressed for delivery to a target site. In some instances, the implant device includes an attachment feature(s) configured for expansion to attach/anchor/seal the implant device to the native tissue. However, the implant device can be configured in other manners.

At block 1204, the process 1200 includes sizing and/or selecting the implant device. For example, an aneurysm/aneurysmal portion of the aorta can be measured using one or more imaging techniques, such as x-rays, fluoroscopy, ultrasound, etc., to capture one or more images of the internal anatomy of a patient including the aneurysmal portion of the aorta. The one or more images can be analyzed/evaluated to determine a size/dimension of the aneurysm/aneurysmal portion, such as a length, width, diameter, or other dimension. In examples, a size/dimension of neighboring health tissue is measured/determined, such as a diameter or other dimension of the aorta within a predetermined distance to the aneurysm/aneurysmal portion.

In examples, an implant device can be cut to a length that satisfies/matches the length of the aneurysm. For instance, one or more ends/attachment features of an implant device can be cut to create the appropriate longitudinal length for the implant device.

Further, in examples, multiple implant devices are available with different sizes/dimensions so that a physician/user can select the most appropriate implant device for the patient/anatomy. To illustrate, attachment segments and/or compliant structures of different implant devices can include different diameters and/or lengths such that a physician/user can select an implant device that satisfies/matches the size/dimension of the aneurysm/aneurysmal portion and/or the size/dimension of neighboring health tissue. For instance, an implant device that has attachment features with a particular amount of oversizing in diameter (or the same/smaller/similar diameter) relative to the diameter of the aorta can be selected.

As such, a measured/determined dimension of anatomy of a patient can be used to determine a device size for an implant device and/or provide/select the appropriate implant device.

At block 1206, the process 1200 includes accessing a fluid vessel that includes an enlargement. For example, a physician/user or robotics system can access an aneurysm/target site in the aorta or another fluid vessel by advancing a delivery system/device that includes the implant device (such as in a compressed configuration/state) to the target site. As such, the implant device can be configured to compress to a delivery/compressed configuration/state for delivery to a target site. In some cases, the delivery system is navigated/guided to the target site using one or more imaging techniques, such as using x-rays, fluoroscopy, ultrasound, etc.

In examples, a delivery system/device can comprise one or more catheters, sheaths, balloons, and/or other devices used to advance and/or implant the implant device, which can be disposed at least partially within the delivery system during portions of the process 1200. An implant device can be positioned within the delivery system with a first end of the implant device disposed proximally relative to the delivery system and a second end disposed distally with respect to the delivery system. In some cases, the second end that is disposed distally includes a prosthetic valve.

In examples, a delivery system comprises an outer catheter/shaft/sheath, which can be used to transport an implant device to the target implantation site. That is, the implant device can be advanced to the target implantation site at least partially within a lumen of the outer shaft, such that the implant device is held and/or secured at least partially within a distal portion of the outer shaft in a radially compressed configuration.

In examples, a delivery system comprises a tapered nosecone feature, which can facilitate advancement of the distal end of the delivery system through the tortuous anatomy of the patient and/or an outer delivery sheath or other conduit/path. The nosecone can be a separate component from the outer shaft or can be integrated with the outer shaft. In some examples, the nosecone is adjacent to and/or integrated with a distal end of the outer shaft. In some examples, the nosecone is distally tapered into a generally conical shape and can comprise and/or be formed of multiple flap-type forms that can be urged/spread apart when the implant device and/or any portions thereof, interior shafts, or devices, are advanced distally therethrough.

A delivery system can further be configured to have a guidewire disposed at least partially within the delivery system. The guidewire can provide a path through the patient from an external surface/port to the target site within the anatomy, such as from a percutaneous access point/port or natural orifice to an aneurysm/target site. In some implementations, the guidewire can pass through an interior of the implant device and/or through a lumen of a pusher device or tube of the delivery system.

At block 1208, the process 1200 includes deploying/implanting the implant device, such as from a delivery system. In examples, to deploy the implant device, an outer sheath of the delivery device is proximally pulled and/or a pusher (located proximally relative to the delivery system) is distally pushed to thereby draw the outer sheath past the distal end of the implant device, at least partially exposing/deploying the implant device. Initially the outer sheath can be withdrawn to position/attach a first attachment feature of the implant device at a first position/location within the aorta (e.g., attach the first attachment feature to a first internal portion of the aorta at one side of the aneurysm). The outer sheath can be further withdrawn to position a compliant segment of the implant device within the aneurysm. The outer sheath can be further withdrawn to position/attach a second attachment feature of the implant device at a second/proximal position/location within the aorta (e.g., attach the second attachment feature to a second internal portion of the aorta at a second side of the aneurysm). The implant device can comprise one or more radiopaque markers that can be referenced to determine/confirm the position of the implant device at various stage(s) of the process 1200 using a suitable imaging technique.

At block 1210, the process 1200 includes expanding the implant device. In some examples, one or more portions of the implant device (e.g., attachment features, a compliant segment, etc.) are self-expandable, such that implant device is expanded/fully deployed (e.g., anchored/secured to the internal tissue of the aorta) upon release from the outer sheath (as discussed above with reference to block 1208). For instance, expansion of an attachment feature and/or a compliant segment can be achieved via shape memory features of the attachment feature, compliant segment, and/or other portions of the device. To illustrate, one or more portions of the implant device (e.g., one or more frames of the device) can comprise nitinol or another shape-memory metal configured to self-expand when released from the delivery sheath/capsule.

Alternatively, or additionally, one or more portions of the implant device (e.g., attachment features and/or a compliant segment) can be balloon/dilator expandable, such that a balloon/dilator are used to radially expand the component. The balloon/dilator can be implemented as part of or separately from the delivery system. The balloon/dilator can be inserted through the implant device to radially expand one or more portions of the implant device.

At block 1212, the process 1200 includes withdrawing the delivery system, leaving the implant device implanted. The delivery system can be withdrawn from the patient over a guidewire and/or the guidewire can be withdrawn.

As such, the process 1200 can utilize a transcatheter procedure for implantation/deployment of implant devices in accordance with aspects of the present disclosure. However, implant devices disclosed herein can be implanted using other types of minimally invasive and/or surgical procedures.

In examples, once implanted through any process discussed herein, an implant device, such as the device 200, can typically be configured in the non-expanded state (with a smaller cross-sectional area) having a more lobular shape during a lower-pressure period (e.g., diastole). As luminal/radial pressure is exerted/increases within the implant device, a compliant segment of the device can change form, such that a deflectable portion(s) deflects radially outward to form a more circular shape with a larger cross-sectional area. As such, the implant device can exhibit/produce a larger volume during a period/phase of higher pressure (e.g., of a cardiac cycle), and exhibit/produce a smaller volume during a period/phase of lower pressure. The change in shape/form/cross-sectional area can result in a change in volume of the implant device, thereby mimicking compliance of a native vessel.

In other words, due to an area of a lobular/non-expanded configuration being less than an area of a more circular/rounded/expanded configuration, transitioning from the lobular shape to the more circular shape can provide an increase in area/volume of the implant device. Further, transiting back from the more circular shape to the lobular shape can provide a reduction in area/volume of the implant device. Such changes can provide compliance characteristics. For example, the implant device can transition between lobular and expanded shapes in response to the typical changes in pressure experienced during the cardiac cycle, which can introduce volumetric change in the implant device, thereby increasing cardiac efficiency, reducing pulsatile load, etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### ADDITIONAL EXAMPLES

Example 1: An implant device comprising: a tubular frame having a biased shape that includes multiple lobes and multiple deflectable walls that are disposed between the multiple lobes and that deflect radially inward, the multiple deflectable walls being configured to deflect radially outward based on luminal pressure.

Example 2: The implant device of any example herein, in particular example 1, wherein the implant device includes a first cross-sectional area when the tubular frame is configured in the biased shape and a second cross-sectional area when the tubular frame is configured in an expanded shape, the expanded shape including the multiple deflectable walls deflected radially outward.

Example 3: The implant device of any example herein, in particular example 1 or 2, further comprising: a covering coupled to at least one of an inner or outer surface of the tubular frame.

Example 4: The implant device of any example herein, in particular examples 1-3, further comprising: an attachment feature coupled to or integral with an end of the tubular frame and configured to attach the implant device to a fluid vessel.

Example 5: The implant device of any example herein, in particular example 4, wherein the attachment feature includes a circular cross-sectional shape.

Example 6: The implant device of any example herein, in particular example 4, wherein the attachment feature includes a cloth.

Example 7: The implant device of any example herein, in particular example 4, wherein the attachment feature includes an expandable frame configured to expand radially.

Example 8: The implant device of any example herein, in particular examples 1-7, wherein the multiple lobes and multiple deflectable walls extend longitudinally.

Example 9: The implant device of any example herein, in particular examples 1-8, further comprising: a prosthetic valve coupled to an end portion of the tubular frame.

Example 10: The implant device of any example herein, in particular examples 1-9, wherein the tubular frame is configured to flex longitudinally.

Example 11: The implant device of any example herein, in particular examples 1-10, wherein the tubular frame includes a shape-memory metal.

Example 12: The implant device of any example herein, in particular examples 1-11, wherein the implant device is configured to be compressed and coupled to a delivery system.

Example 13: The implant device of any example herein, in particular examples 1-12, wherein the tubular frame includes one or more first frames that form the multiple lobes and one or more second frames that form the multiple deflectable walls.

Example 14: The implant device of any example herein, in particular examples 1-13, wherein the multiple lobes are formed of a first material and the multiple deflectable walls are formed of a second material that is different than the first material.

Example 15: The implant device of any example herein, in particular examples 1-14, wherein the implant device is sterilized.

Example 16: An implant device comprising: a tubular structure with a cross-sectional shape having first sections that deflect radially inward and are separated by second sections, the first sections being configured to deflect radially outward based on radial pressure; and an attachment feature coupled to or integral with the tubular structure and configured to couple to a fluid vessel.

Example 17: The implant device of any example herein, in particular example 16, wherein the first sections are biased towards a first state in which the first sections are deflected radially inward.

Example 18: The implant device of any example herein, in particular example 17, wherein the implant device includes a first cross-sectional area for the first state and a second cross-sectional area for a second state in which the first sections are deflected radially outward, the first cross-sectional area being smaller than the second cross-sectional area.

Example 19: The implant device of any example herein, in particular examples 16-18, further comprising: a covering coupled to at least one of an inner or outer surface of the tubular structure.

Example 20: The implant device of any example herein, in particular examples 16-19, wherein the attachment feature includes a circular cross-sectional shape.

Example 21: The implant device of any example herein, in particular examples 16-20, wherein the attachment feature includes a cloth.

Example 22: The implant device of any example herein, in particular examples 16-21, wherein the attachment feature includes an expandable frame configured to expand radially.

Example 23: The implant device of any example herein, in particular examples 16-22, wherein the first sections and second sections extend longitudinally.

Example 24: The implant device of any example herein, in particular examples 16-23, further comprising: a prosthetic valve coupled to an end portion of the tubular structure.

Example 25: The implant device of any example herein, in particular examples 16-24, wherein the tubular structure is configured to flex longitudinally.

Example 26: The implant device of any example herein, in particular examples 16-25, wherein the tubular structure includes a shape-memory metal.

Example 27: The implant device of any example herein, in particular examples 16-26, wherein the implant device is configured to be compressed and coupled to a delivery system.

Example 28: The implant device of any example herein, in particular examples 16-27, wherein the tubular structure includes one or more first frames that form the first sections and one or more second frames that form the second sections.

Example 29: The implant device of any example herein, in particular examples 16-28, wherein the first sections are formed of a first material and the second sections are formed of a second material that is different than the first material.

Example 30: The implant device of any example herein, in particular examples 16-29, wherein the implant device is sterilized.

Example 31: A method comprising: providing an implant device that includes a tubular structure with a cross-sectional shape having first sections that deflect radially inward and that are separated by second sections, the first sections being configured to deflect radially outward based on radial pressure; accessing a fluid vessel that includes an enlargement, a first location on one side of the enlargement, and a second location on another side of the enlargement; and implanting the implant device in the fluid vessel by: attaching a first end portion of the implant device to the first location; and attaching a second end portion of the implant device to the second location.

Example 32: The method of any example herein, in particular example 31, wherein the implanting the implant device includes suturing the first end portion to the first location and suturing the second end portion to the second location.

Example 33: The method of any example herein, in particular example 31 or 32, further comprising: resecting the fluid vessel at the enlargement before or after implanting the implant device.

Example 34: The method of any example herein, in particular examples 31-33, further comprising: determining a size of the enlargement; determining a device size based at least in part on the size of the enlargement; and cutting the implant device to the device size.

Example 35: The method of any example herein, in particular examples 31-34, wherein the enlargement is an aneurysm and the fluid vessel is a blood vessel.

Example 36: The method of any example herein, in particular examples 31-35, wherein the fluid vessel is the aorta.

Example 37: The method of any example herein, in particular examples 31-36, wherein the first sections are biased towards a first state in which the first sections are deflected radially inward.

Example 38: The method of any example herein, in particular example 37, wherein the implant device includes a first cross-sectional area for the first state and a second cross-sectional area for a second state in which the first sections are deflected radially outward, the first cross-sectional area being smaller than the second cross-sectional area.

Example 39: The method of any example herein, in particular examples 31-38, wherein the implant device further includes a covering coupled to at least one of an inner or outer surface of the tubular structure.

Example 40: The method of any example herein, in particular examples 31-39, wherein the first end portion of the implant device includes an expandable frame.

Example 41: The method of any example herein, in particular examples 31-40, wherein the first end portion of the implant device includes a circular cross-sectional shape.

Example 42: The method of any example herein, in particular examples 31-41, wherein the first end portion of the implant device includes a cloth.

Example 43: The method of any example herein, in particular examples 31-42, wherein the first sections and the second sections extend longitudinally.

Example 44: The method of any example herein, in particular examples 31-43, wherein the implant device further includes a prosthetic valve.

Example 45: The method of any example herein, in particular examples 31-44, wherein the tubular structure is configured to flex longitudinally.

Example 46: The method of any example herein, in particular examples 3145, wherein the tubular structure includes a shape-memory metal.

Example 47: The method of any example herein, in particular examples 31-46, wherein the implant device is configured to be compressed and coupled to a delivery system.

Example 48: The method of any example herein, in particular examples 3147, wherein the tubular structure includes one or more first frames that form the first sections and one or more second frames that form the second sections.

Example 49: The method of any example herein, in particular examples 31-48, wherein the first sections are formed of a first material and the second sections are formed of a second material that is different than the first material.

Example 50: The method of any example herein, in particular examples 31-49, wherein the implant device is sterilized.

Example 51: The method of any example herein, in particular examples 31-50, further comprising: covering at least a portion of the implant device with a portion of the fluid vessel that has been resected.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that some examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, can encompass the concepts of equality. For example, “less” can mean not only “less” in the strictest mathematical sense, but also “less than or equal to.”

What is claimed is:

1. An implant device for providing compliance to a blood vessel, the implant device comprising:
a radially expandable and collapsible tubular frame, the tubular frame having a cross-section defining three lobed portions in a collapsed state, wherein each of the lobed portions extends farther radially outwardly than intermediate portions positioned between the lobed portions and wherein the intermediate portions of the tubular frame move radially outwardly to form a more circular cross-section in an expanded state; and
a cover extending over the tubular frame for providing a seal;
wherein the tubular frame expands and collapses with changes in blood pressure for providing additional compliance to the blood vessel.

2. The implant device of claim 1, wherein the implant device includes a first cross-sectional area when the tubular frame is in the collapsed state and a second cross-sectional area when the tubular frame is in an expanded state, the second cross-sectional area being larger than the first cross-sectional area.

3. The implant device of claim 1, further comprising:
an attachment feature coupled to or integral with an end of the tubular frame and adapted to attach the implant device to a fluid vessel.

4. The implant device of claim 3, wherein the attachment feature includes a circular cross-sectional shape.

5. The implant device of claim 1, wherein the lobed portions extend longitudinally.

6. The implant device of claim 1, further comprising:
a prosthetic valve coupled to an end portion of the tubular frame.

7. An implant device comprising:
a tubular structure with a cross-sectional shape having first sections that are separated by second sections, the tubular structure including a first cross-sectional area for a first state and a second cross-sectional area for a second state, the first state including the first sections deflected radially inward and the second state including the first sections deflected radially outward; and
an attachment feature coupled to or integral with the tubular structure and dimensioned to couple to a fluid vessel, the attachment feature including a circular cross-sectional shape.

8. The implant device of claim 7, wherein the first cross-sectional area is smaller than the second cross-sectional area.

9. The implant device of claim 7, further comprising:
a covering coupled to at least one of an inner or outer surface of the tubular structure.

10. The implant device of claim 7, wherein the tubular structure includes a shape-memory metal.

11. The implant device of claim 7, wherein the attachment feature includes a cloth.

12. The implant device of claim 7, wherein the attachment feature includes an expandable frame adapted to expand radially.

13. The implant device of claim 7, wherein the first sections and second sections extend longitudinally.

14. The implant device of claim 7, wherein the tubular structure is adapted to flex longitudinally.

15. An implant device comprising:
a tubular frame having a biased shape that includes multiple lobes and multiple deflectable walls that are disposed between the multiple lobes, the multiple deflectable walls being adapted to deflect radially outward based on luminal pressure, the multiple lobes including a first lobe and a second lobe that are positioned at different angular locations around a common axis.

16. The implant device of claim 15, wherein the tubular frame includes a shape-memory metal.

17. The implant device of claim 15, wherein the implant device is adapted to be compressed and coupled to a delivery system.

18. The implant device of claim 15, wherein the tubular frame includes one or more first frames that form the multiple lobes and one or more second frames that form the multiple deflectable walls.

19. The implant device of claim 15, wherein the multiple lobes are formed of a first material and the multiple deflectable walls are formed of a second material that is different than the first material.

20. The implant device of claim 15, wherein the implant device is sterilized.
